(19) United States
(12) Patent Application Publication
(10) Pub. No.: US 2009/0075380 A1
(43) Pub. Date: Mar. 19, 2009

(54) Methods and Compositions To Enhance Efficiency Of Nuclear Transfer/Cloning

(75) Inventors: George W. Smith, Onondaga, MI (US); Kyungbon Lee, Lansing, MI (US); Catherine VandeVoort, El Macero, CA (US)

Correspondence Address: Peter G. Carroll;MEDLEN & CARROLL, LLP, Suite 350, 101 Howard Street, San Francisco, CA, 94105, US

(73) Assignee: Michigan State University System

(21) Appl. No.: 12/176,863

(22) Filed: Jul. 21, 2008

Related U.S. Application Data
(60) Provisional application No. 60/961,196, filed on Jul. 19, 2007.

(51) Int. Cl. C12N 5/10; C12N 5/02; C12N 15/00

(52) U.S. Cl. 435/378; 435/404; 800/24

(57) ABSTRACT

The present invention provides compositions and methods for increasing the success of assisted reproductive technology (ART). Specifically, the inventions described herein increase the survival rate of manipulated embryos for increasing post implantation numbers of viable offspring. In particular, the present invention provides for compositions and methods for allowing further embryonic development and increasing rates of embryonic maturation, such as increasing cleavage rate, TE numbers, and blastocyte formation of in vitro fertilized and nuclear transfer embryos in media comprising follistatin, thereby providing for increased survival of fertilized and manipulated embryos leading to increased numbers of live offspring from in vitro fertilized and implanted nuclear transfer embryos. Further provided are diagnostic kits for determining transplantation potential.

### FIELD OF THE INVENTION

The present invention provides compositions and methods for increasing the success of assisted reproductive technology (ART). Specifically, the inventions described herein increase the survival rate of manipulated embryos for increasing post implantation numbers of viable offspring. In particular, the present invention provides for compositions and methods for allowing further embryonic development and increasing rates of embryonic maturation, such as increasing cleavage rate, TE numbers, and blastocyte formation of in vitro fertilized and nuclear transfer embryos in media comprising follistatin, thereby providing for increased survival of fertilized and manipulated embryos leading to increased numbers of live offspring from in vitro fertilized and implanted nuclear transfer embryos. Further provided are diagnostic kits for determining transplantation potential.

### BACKGROUND OF THE INVENTION

Poor oocyte competence contributes to infertility in humans and livestock species. Two well-defined bovine models of oocyte competence are the prepubertal oocyte and time to first cleavage models. Bovine embryos produced in vitro from oocytes harvested from prepubertal animals show reduced development to the blastocyst stage (Revel et al., 1995, J. Reprod. Fertil. 103:115-120; Damiani et al., 1996, Mol. Repro. Dev. 45:521-534; all of which are herein incorporated by reference) and transfer of in vitro (Khatir et al., 1998, Theriogenology 50:1201-1210) and in vivo produced bovine embryos (Armstrong et al., 2001, Theriogenology 55:1303-1322; all of which are herein incorporated by reference) derived from oocytes of prepubertal animals results in reduced pregnancy success. Furthermore, a higher proportion of bovine embryos that cleave early (e.g., 30 h post fertilization), rather than late (e.g., 36 h post fertilization) reach the blastocyst stage (Plante et al., 1994, Mol. Repro. Dev. 39:375-383; Lonergan et al., 1999, J. Repro. Fertil. 117:159-167; all of which are herein incorporated by reference).

During the initial cleavage divisions post-fertilization, embryonic development is supported by maternal mRNAs and proteins synthesized and stored during oogenesis which are critical for the interval between fertilization and the maternal-embryonic transition when transcriptional activity of the embryonic genome becomes robust at the 8-16 cell stage in cattle (Telford et al., 1990, Mol. Repro. Dev. 26:90-100; De Sousa et al., 1998, Mol. Repro. Dev. 51:112-121; all of which are herein incorporated by reference). Thus, time to first cleavage of early bovine embryos is likely significantly influenced by differences in oocyte derived mRNA and proteins. However, the cloning/nuclear transfer technology for propagation of valuable livestock species, generation of transgenic animals, therapeutic cloning and research purposes is currently limited by low efficiency.

As such, what are needed are compositions and methods for increasing the efficiency of assisted reproductive technologies. In particular increased rates of blastocyte and trophectoderm formation are needed for cultured embryos.

### SUMMARY OF THE INVENTION

The present invention provides compositions and methods for increasing the success of assisted reproductive technology (ART). Specifically, the inventions described herein increase the survival rate of manipulated embryos for increasing post implantation numbers of viable offspring. In particular, the present invention provides for compositions and methods for allowing further embryonic development and increasing rates of embryonic maturation, such as increasing cleavage rate, TE numbers, and blastocyte formation of in vitro fertilized and nuclear transfer embryos in media comprising follistatin, thereby providing for increased survival of fertilized and manipulated embryos leading to increased numbers of live offspring from in vitro fertilized and implanted nuclear transfer embryos. Further provided are diagnostic kits for determining transplantation potential.

In one embodiment, the present invention contemplates a culture medium for in vitro culture of embryos comprising follistatin, wherein the development and survival of said embryos is enhanced when grown in said culture media compared to growth in culture media without follistatin. In one embodiment, the follistatin is present at a concentration from about 1 ng/ml to about 20 ng/ml. In one embodiment, the follistatin is present at a concentration of about 10 ng/ml. In one embodiment, the method further comprises an embryo, wherein said embryo is selected from the group consisting of in vitro fertilization embryos, nuclear transfer embryos, cloned embryos, noncloned embryos, embryos for assisted reproductive techniques. In one embodiment, the embryos are mammalian embryos.

In one embodiment, the present invention contemplates a kit comprising a culture medium and mammalian embryos. In one embodiment, the kit further comprises a sheet of instructions.

In one embodiment, the present invention contemplates a method of increasing the survival of embryos, comprising, a) providing, i) an embryo, ii) a culture medium, wherein said culture medium comprises follistatin, b) culturing said embryo in a culture medium wherein the survival of said embryo is increased compared to survival of an embryo not grown in said media. In one embodiment, the embryo is an in vitro fertilization embryo. In one embodiment, the nuclear transfer embryo is a nuclear transfer embryo. In one embodiment, the nuclear transfer embryo comprises genetic material obtained from a somatic cell. In one embodiment, the follistatin is present at a concentration from about 1 ng/ml to about 20 ng/ml. In one embodiment, the follistatin is present at a concentration of about 10 ng/ml. In one embodiment, the increased survival of said embryo is increasing the number of trophectoderm cells. In one embodiment, the follistatin is a human recombinant follistatin. In one embodiment, the method further comprises obtaining a biopsy from said embryo. In one embodiment, the method further comprises c) determining the amount of follistatin expression in said embryo.

In one embodiment, the present invention contemplates a method of generating stem cells comprising: a) providing, i) an embryo, ii) a culture medium, wherein said culture medium comprises follistatin, b) culturing said embryo in a culture medium to provide cultured embryonic cells, c) generating stem cells from said cultured embryonic cells. In one embodiment, the culture of said embryo in said media generates an increased number of stem cells from said embryo as compared to the number of stem cells generated from an embryo not grown in said culture medium. In one embodiment, the method further comprises step d) harvesting said stem cells. In one embodiment, the follistatin is present at a concentration from about 1 ng/ml to about 20 ng/ml.

Other illustrative embodiments of the invention are described below. The present invention is not limited to these embodiments.

In some embodiments, the present invention provides compositions and methods for maintenance/growth of nuclear transfer cloned embryos wherein said maintenance/growth favors the survival of said embryos, the development of trophectoderm cells in a blastocyst, and increases the numbers of live offspring from said embryos. As such, methods and compositions of the present invention provide a wide range of applications, including but not limited to applications to the commercial cloning industry for livestock and animal biotechnology industries, human regenerative medicine and research applications including noncloned and cloned embryos.

In some embodiments, the present invention provides for the development of culture medium comprising, for example, a compound of interest and the development of tools/diagnostics to select eggs with high levels of endogenous follistatin. In some embodiments, the compound of interest is follistatin. In some embodiments, follistatin is added to already existing culture media as defined in U.S. Pat. Nos. 5,096,822, 5,563,059, and 5,693,534, all incorporated by reference herein in their entireties. In some embodiments, the present invention provides for the culture of nuclear transfer embryos in the above-mentioned culture media thereby increasing the viability of said embryos and offspring resulting from said embryos.

In some embodiments, the present invention provides methods for selecting oocytes for nuclear transfer of genetic material for nuclear transfer cloning comprising measuring the level of endogenous follistatin in said oocytes and selecting for oocytes with increased levels of endogenous follistatin for nuclear transfer of genetic material (e.g., DNA).

In some embodiments, the present invention provides for culture media for in vitro culture of embryos, in some embodiments nuclear transfer embryos, comprising follistatin, wherein the development and survival of said embryos is enhanced when grown in said culture media compared to growth in culture media without follistatin. In some embodiments the follistatin is in the culture media at a concentration of about 1 ng/ml to about 20 ng/ml. In some embodiments the follistatin is in the culture media at a concentration of about 10 ng/ml. In some embodiments, the embryo cultured in the culture medium supplemented with follistatin are derived from groups including but not limited to in vitro fertilization embryos, nuclear transfer embryos, cloned embryos, noncloned embryos, embryos for assisted reproductive techniques, and the like.

In some embodiments, the present invention provides methods for increasing the survival of nuclear transfer embryos comprising providing embryos comprising nuclear transferred genetic material, such as from in vitro fertilization, nuclear transfer cloning, somatic cells, and the like, growing and maintaining the embryos in a culture media comprising follistatin of concentrations of about 1 ng/ml to 20 ng/ml wherein the survival of the embryos is increased compared to survival of embryos not grown in the follistatin supplemented media. In some embodiments, the nuclear transferred genetic material is somatic cell genetic material.

In further embodiments, the present invention provides methods for increasing the number of trophectoderm cells in a nuclear transfer embryo derived blastocyst comprising providing embryos comprising nuclear transferred genetic material and growing the embryos in follistatin supplements culture media at a concentration of about 1 ng/ml to about 20 ng/ml, and increasing the number of trophectoderm cells when the embryos are grown in the follistatin supplemented media compared to embryos grown in non-follistatin supplemented media. In some embodiments, the cultured embryos are derived from in vitro fertilization or nuclear transfer cloning. In some embodiments, the nuclear transferred genetic material in the embryos is somatic cell material.

In some embodiments, the present invention provides methods for the generation of stem cells comprising providing somatic cell nuclear transfer embryos, culturing the embryos in a culture media comprising follistatin at a concentration of, for example, about 1 ng/ml to about 20 ng/ml and deriving the stem cells from the cultured embryonic cells. In some embodiments, the culture of the somatic cell nuclear transfer embryos in follistatin-supplemented media generates an increased number of stem cells compared to embryos grown in non-follistatin supplemented media. In some embodiments, the methods of the present invention further provide for the harvesting of the stem cells from embryos grown in follistatin-supplemented media.

In some embodiments, the present invention provides methods for increasing transplantation potential, comprising, providing, an embryo, a medium comprising follistatin, incubating said embryo in said medium, determining the amount of expressed follistatin. In one embodiment, the embryo provides a biopsy for determining the amount of follistatin. In one embodiment, the biopsy is evaluated for the amount of expressed follistatin. In one embodiment, amount of expressed follistatin is follistatin mRNA. In one embodiment, the amount of expressed follistatin is follistatin protein. In one embodiment, the amount of expressed follistatin is determined using an antifollistatin antibody.

In some embodiments, the present inventions provide a diagnostic kit for determining embryonic transplantation potential. In one embodiment, the kit comprises detection reagents for determining embryonic transplantation potential, including but not limited to reagents for detecting follistatin mRNA expression, an antifollistatin antibody, and the like.

### DESCRIPTION OF THE FIGURES

FIG. 1 shows an exemplary demonstration of an effect of follistatin supplementation during the first 72 h of in vitro culture of bovine embryos on the time to first cleavage. Effect of follistatin treatment on proportion of embryos that reached the 2-cell stage (A) within 30 h post insemination (early cleaving), and (B) from 30-36 h post insemination (late cleaving), Values are expressed as mean±SEM of the data collected from six replicates. Values with different superscripts across treatments indicate significant differences (P<0.05).

FIG. 2 shows an exemplary demonstration of an effect of follistatin supplementation during the first 72 h of in vitro culture of bovine embryos on development to the blastocyst stage (day 7). The values are expressed as mean±SEM of the data collected from six replicates. Values with different superscripts across treatments indicate significant differences (P<0.05).

FIG. 3 shows an exemplary demonstration of an effect of follistatin supplementation on rate of development of early versus late cleaving bovine embryos to the blastocyst stage in vitro. Effects of exogenous follistatin on (A) proportion of early cleaving embryos reaching the blastocysts stage and (B) proportion of late cleaving embryos reaching the blastocyst stage are depicted. The values are expressed as mean±SEM of the data collected from four replicates. Values with different superscripts across treatments indicate significant differences (P<0.05).

FIG. 4 shows an exemplary demonstration of an effect of follistatin supplementation during the first 72 h of in vitro culture of bovine embryos on day 7-blastocyst cell allocation. Effect of exogenous follistatin supplementation on (A) the number of inner cell mass (ICM) cells, (B) number of trophectoderm (TE) cells and (C) total cell numbers are depicted. The total number of blastocysts examined for staining after 0, 1, 10 and 100 ng/ml follistatin treatment was 14, 22, 27 and 15, respectively. The values are expressed as mean±SEM of the data collected from four replicates. Values with different superscripts across treatments indicate significant differences (P<0.05).

FIG. 5 shows an exemplary demonstration of an effect of follistatin supplementation during first 72 h of in vitro culture of bovine embryos on % ICM cells and TE cells in day 7 blastocyst stage embryos. Effect of exogenous follistatin supplementation on (A) the proportion of ICM cells versus total cells and (B) the proportion of TE cells versus total cells are depicted. The values are expressed as mean i SEM of the data collected from four replicates. Values with different superscripts across treatments indicate significant differences (P<0.05).

FIG. 6 shows an exemplary demonstration of an effect of follistatin supplementation during first 72 h of in vitro culture of bovine embryos on the proportion of embryos reaching blastocyst stage (day 7) with different ICM/total cell ratios (ICM ratio) reflective of embryo quality. Effects of exogenous follistatin supplementation on proportions of day 7 blastocysts with (A) ICM ratio of 20-40%, (B) ICM ratio of 40-60% and (C) ICM ratio of >60% are depicted. The values are expressed as mean±SEM of the data collected from four replicates. Values with different superscripts across treatments indicate significant differences (P<0.05).

FIG. 7 shows an exemplary demonstration of A) an effect of follistatin short interfering RNA (siRNA) knockdown of follistatin mRNA and protein in bovine in vitro fertilized embryos in vitro and B) an effect of microinjection of follistatin siRNA on follistatin protein abundance in 16-cell embryos in vitro. The results are expressed as mean±SEM of the data collected from four replicates. Values with different superscripts across treatments indicate significant differences (P<0.05).

FIG. 8 shows an exemplary demonstration of an effect of microinjection of follistatin siRNA on development of in vitro fertilized bovine embryos; A) early cleaving embryos, B) 8-16 cell embryos, and C) blastocysts. The results are expressed as mean±SEM of the data collected from four replicates. Values with different superscripts across treatments indicate significant differences (P<0.05).

FIG. 9 shows an exemplary demonstration of the effect of follistatin treatment on development of nuclear transfer embryos to the blastocyst stage. The results are expressed as mean±SEM of the data collected from four replicates. Values with different superscripts across treatments indicate significant differences (P<0.05).

FIG. 10 shows an exemplary demonstration of an effect of follistatin treatment on cell allocation in nuclear transfer blastocysts. The effect of follistatin treatment on number of trophectoderm cells is depicted. The number of blastocysts examined for staining were 22 (parthenogenetic embryo control), 17 (0 ng/ml follistatin), 23 (1 ng/ml follistatin), 26 (10 ng/ml follistatin) and 15 (100 ng/ml follistatin). The values are expressed as mean±SEM of the data collected from four replicates. Values with different superscripts across treatments indicate significant differences (P<0.05).

FIG. 11 shows an exemplary demonstration of an effect of follistatin supplementation during first 72 h of in vitro culture of bovine embryos on % ICM cells and TE cells in day 7 blastocyst stage embryos generated via nuclear transfer. Effect of exogenous follistatin supplementation on (A) the proportion of ICM cells versus total cells and (B) the proportion of TE cells versus total cells are depicted. The values are expressed as mean±SEM of the data collected from four replicates. Values with different superscripts across treatments indicate significant differences (P<0.05).

FIG. 12 shows an exemplary demonstration of an effect of follistatin supplementation during first 72 h of in vitro culture of bovine nuclear transfer embryos on the proportion of embryos reaching blastocyst stage (day 7) with different ICM/total cell ratios (ICM ratio) reflective of embryo quality. No significant effect of follistatin treatment on proportions of day 7 nuclear transfer blastocysts with ICM ratio of 20-40% or 40-60% was observed. The values are expressed as mean±SEM of the data collected from four replicates. Values with different superscripts across treatments indicate significant differences (P<0.05).

FIG. 13 shows an exemplary demonstration of an effect of follistatin ablation (siRNA microinjection) and (or) replacement (follistatin supplementation) on bovine embryonic development. Presumptive zygotes were microinjected (16-18 h post-fertilization) with approximately 20 pl of follistatin siRNA or served as uninjected controls. Uninjected or injected presumptive zygotes were cultured serum free in KSOM medium supplemented with 0.3% BSA and with or without 10 ng/ml follistatin (FS) maximally effective dose determined in previous studies; 25-30 presumptive zygotes per group). The 8-16 cell stage embryos were then separated and cultured in fresh KSOM medium supplemented with 0.3% BSA and 10% FBS in absence of exogenous follistatin until d 7. Effect of follistatin ablation and (or) replacement on (Top) proportion of embryos developing to the 8-16 cell stage and (Bottom) proportion of embryos developing to blastocyst stage. Data are expressed as mean±SEM from four replicates. Values with different superscripts across treatments indicate significant differences (P<0.05).

FIG. 14 shows an exemplary demonstration of an effect of follistatin ablation (siRNA microinjection) and (or) replacement [follistatin (FS) supplementation] on cell allocation within bovine blastocysts (d 7). Number of inner cell mass (ICM), trophectoderm (TE) and total cell numbers were determined by cell counts on differentially stained embryos (Machaty et al., 1998; herein incorporated by reference). Effect of follistatin ablation and (or) replacement on (Top) number of ICM cells, (Middle) number of TE cells and (Bottom) total cell numbers are recorded. Data are expressed as mean±SEM from four replicates. Values with different superscripts across treatments indicate significant differences (P<0.05).

FIG. 15 shows A) an exemplary demonstration of an effect of follistatin ablation (siRNA microinjection) and (or) replacement [follistatin (FS) supplementation] on mRNA abundance for the inner cell mass marker Nanog (top) and trophectoderm (TE) cell marker CDX-2 (bottom) in bovine blastocysts. Blastocysts were harvested at d 7 post-fertilization (n=4 pools of 2 blastocysts each per treatment) and subjected to RNA isolation and quantitative real-time RT-PCR analysis of mRNA abundance for Nanog and CDX-2. Data were normalized relative to abundance of endogenous control (18S rRNA) and are shown as mean±SEM. Means without common superscripts in each panel are significantly different (P<0.05). B) an exemplary schematic diagram of where Nanog and CDX-2 influence cell fate in a developing embryo (Duranthon, et al., Reproduction (2008) 135 141-150, FIG. 2; herein incorporated by reference).

FIG. 16 shows an exemplary demonstration of an effect of follistatin supplementation (10 ng/ml) during initial 48 h of in vitro culture of rhesus monkey embryos on (Top) % cleavage at 30 h post insemination and (Bottom) % development to the blastocyst stage (determined on d 8). (a,b; P<0.05).

### DEFINITIONS

As used herein, the term “subject” refers to any animal (e.g., a mammal), including, but not limited to, humans, non-human primates, rodents, and the like, which is to be the recipient of a particular treatment.

As used herein, the term “non-human animals” refers to all non-human animals including, but are not limited to, livestock, vertebrates such as rodents, non-human primates, ovines, bovines, ruminants, lagomorphs, porcines, caprines, equines, canines, felines, aves, etc.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

The term “transgene” as used herein refers to a foreign gene that is placed into an organism by, for example, introducing the foreign gene into newly fertilized eggs or early embryos, thereby producing, for example, a “transgenic animal”. The term “foreign gene” refers to any nucleic acid (e.g., gene sequence) that is introduced into the genome of an animal by experimental manipulations and may include gene sequences found in that animal so long as the introduced gene does not reside in the same location, as does the naturally occurring gene.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro. Cells cultured in the present application include oocytes, embryos and embryo derived cell masses.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide.

The term “transgene” as used herein refers to a foreign, heterologous, or autologous gene that is placed into a cell or an organism by introducing the gene into newly fertilized eggs or early embryos, such as a gene encoding an siRNA of the present inventions. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in an embryo, a host cell may be a zygote.

As used herein, the term “somatic cell” in general refers to any diploid mammalian cell, such as a fibroblast, and the like, with the exception of a fertilized egg (i.e. zygote).

As used herein, the term “gamete” or “germ cell” in reference to a cell refers to cell consisting of a single (haploid) set of chromosomes. Specifically, a gamete is selected from a sperm cell (a spermatocyte or spermatozoa) and an egg cell (oocyte), in other words a “reproductive cell.”

As used herein, the term “ploidy” refers to the number of sets of chromosomes within a cell or an organism. For example, haploid means one set of chromosomes and diploid means two sets of chromosomes.

As used herein, the term “cell differentiation” in general refers to a progressive restriction of developmental potential and increasing specialization of function of cells that takes place during progressive stages of development of the embryo which leads to the formation of specialized cells, tissues, and organs.

As used herein, the term “totipotent” in general refers to an embryonic cell capable of giving rise to all types of differentiated cell found in that organism. In other words, a single totipotent cell could, by division, reproduce a whole organism, for example, a zygote.

As used herein, the term “pluripotent” refers to a cell capable of maturing or develop in any of several ways.

As used herein, the term “pluripotent stem cell” refers to a cell whose descendants are capable of developing into many different types of cells or tissues in the body.

As used herein, the term “meiosis” refers to a reductive cell division which results in daughter cells containing one copy of each of the chromosomes of the parent, such as the process that produces a sperm cell and an egg cell. The entire meiotic process involves two separate divisions (Meiosis I (Reduction) and Meiosis II (Division)). Meiosis I and II are both divided into prophase, metaphase, anaphase, and telophase, such as prophase I, prophase II, and the like.

As used herein, the term “mitosis” refers to a process whereby a cell nucleus divides into two daughter nuclei, each having the same genetic component as the parent cell; in other words mitosis refers to nuclear division plus cytokinesis, and produces two identical daughter cells during prophase, prometaphase, metaphase, anaphase, and telophase.

As used herein, the term “cell cycle” refers to a cell while engaged in metabolic activity and performing its preparation for mitosis (prometaphase, metaphase, anaphase, and telophase), where the cell cycle is divided into interphase G1 (GAP 1)-S (DNA synthesis)-G2 (GAP 2)-M (mitotic) stages.

As used herein, the term “cleavage” in reference to an embryo refers to early embryo cleavage from one cell to at least a 2-cell stage.

As used herein, the term “enucleated” refers to a cell from which the nucleus was removed, such as an oocyte used for nuclear transfer to produce a cloned animal from a nucleus from a somatic cell, such as a differentiated cell.

As used herein, the term “stem cell” refers to a Relatively undifferentiated cells of the same lineage (family type) that retain the ability to divide and cycle throughout postnatal life to provide cells that can become specialized

As used herein, the term “stem cell” in reference to an embryo, as in an “embryonic stem cell” refers to totipotent cell.

As used herein, the term “medium” in reference to a liquid or gelatinous substance containing nutrients for culturing a cell or tissue (such as an embryo) refers to a singular form while media refers to a plural form of the substance.

As used herein, the term “stem cell” refers to a generalized “mother” or “parental” cell that has pluripotency, i.e. daughter cells or descendants may specialize in different functions, such as an undifferentiated mesenchymal cell that is a progenitor (stem cell) for both red and white blood cells.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA; many antisense RNAs block the expression of a target gene by interfering with the processing, transport and/or translation of its primary transcript, for example mRNA. The complementarity of an anti sense RNA may be with any part of the specific gene transcript, i.e., at the 5′ non-coding sequence, 3′ non-coding sequence, introns, or the coding sequence. In addition, antisense RNA may contain regions of ribozyme sequences that increase the efficacy of anti sense RNA to block gene expression. “Ribozyme” refers to a catalytic RNA and includes sequence-specific endoribonucleases. “Anti sense inhibition” refers to the production of anti sense RNA transcripts capable of preventing the expression of the target protein, or of preventing the function of a target RNA.

The term “siRNAs” refers to short interfering RNAs. In some embodiments, siRNAs comprise a duplex, or double-stranded region, where each strand of the double stranded region is about 18 to about 25 nucleotides long; the double stranded region can be as short as 16, and as long as 29, base pairs long, where the length is determined by the anti sense strand. Often siRNAs contain from about two to four unpaired nucleotides at the 3′ end of each strand. siRNAs appear to function as key intermediaries in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

siRNAs may also contain additional sequences; non-limiting examples of such sequences include linking sequences, or loops, which connect the two strands of the duplex region. This form of siRNAs may be referred to “si-like RNA,” “short hairpin siRNA,” where the short refers to the duplex region of the siRNA, or “hairpin siRNA.” Additional non-limiting examples of additional sequences present in siRNAs include stem and other folded structures. The additional sequences may or may not have known functions; non-limiting examples of such functions include increasing stability of an siRNA molecule, or providing a cellular destination signal.

The term “target RNA molecule” refers to an RNA molecule to which at least one strand of the short double-stranded region of an siRNA is complementary. Typically, when such complementarity is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes. The term “ds siRNA” refers to a siRNA molecule which comprises two separate unlinked strands of RNA which form a duplex structure, such that the siRNA molecule comprises two RNA polynucleotides.

The term “enhancing the function” when used in reference to an siRNA molecule means that the effectiveness of an siRNA molecule in silencing gene expression is increased. Such enhancements include but are not limited to increased rates of formation of an siRNA molecule, decreased susceptibility to degradation, and increased transport throughout the cell. An increased rate of formation might result from a transcript which possesses sequences which enhance folding or the formation of a duplex strand.

The term “RNA interference” or “RNAi” refers to the silencing or decreasing expression, or inhibition of expression, of gene expression by siRNAs. It is the process of sequence-specific, post-transcriptional gene silencing in animals and plants, initiated by siRNA that is homologous in its duplex region to the sequence of the silenced gene or that is complementary in its duplex region to the transcriptional product of the silenced gene. The gene may be endogenous or exogenous to the organism, present integrated into a chromosome or present in a transfection vector which is not integrated into the genome. The expression of the silenced gene is either completely or partially inhibited. The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes.

The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]; herein incorporated by reference), has been modified by several groups to optimize conditions for particular types of cells. The art is well aware of these numerous modifications.

The term “assisted reproductive technologies” or “ART” refers to any infertility treatment that uses advanced technology to combine sperm and eggs outside the body in a laboratory, including any infertility treatment that uses advanced technology to combine sperm and eggs outside the body in a laboratory in order to increase fertility. Examples include but are not limited to “ICSI” or “Intracytoplasmic Sperm Injection” referring to a technique that allows reproductive specialists to isolate sperm from a male with which to fertilize eggs in vitro. Once fertilization has taken place, the embryo is allowed to develop for a few days and is then implanted into a uterus at the appropriate time of the reproductive cycle, “ZIFT” or “Zygote Intrafallopian Transfer” refers to fertilization taking place before the egg is placed inside of fallopian tubes. A sample of sperm is mixed with harvested eggs. Once fertilization has taken place (creating a zygote) the fertilized egg is implanted surgically into fallopian tubes.

As used herein, the term “nuclear transfer” is a form of cloning. The steps involve removing the DNA from an oocyte (e.g., unfertilized egg), and injecting a nucleus containing the DNA from the individual to be cloned. Typically, the survival of nuclear transferred derived embryos in non-human mammals (e.g., sheep, bovine, etc.) is very inefficient and embryo viability is very low. The term “somatic cell nuclear transfer” refers to the transfer of DNA from a somatic cell or the entire cell itself (e.g., heart cell, skin cell, nerve cell, etc.) into the empty oocyte. The present invention increases the survival of nuclear transfer cloning embryos through development resulting in an increase in number of blastocyst stage embryos and viable offspring or stem cells (e.g., somatic cell nuclear transfer cloning).

As used herein, the term “infertility” refers to a state of being infertile, for example, not being able to conceive, either in vivo or in vitro, or not being able to support development in order to deliver, either naturally or by cesarean, a live child. As used herein, the term “fertility” refers to a state of being fertile, for example, being able to conceive, either in vivo or in vitro, or being able to support development in order to deliver, either naturally or by cesarean, a live child.

As used herein, the term “IVM” or “in-vitro maturation” refers to a maturation of an immature oocyte in vitro attempting to duplicate the natural process that occurs within the follicle in vivo.

As used herein, the term “IVF” or “in-vitro fertilization” refers to fertilization of an oocytes with a sperm outside of an organism. IVF may also refer to a technique, whereby oocytes and spermatozoa are mixed in the laboratory to achieve fertilization.

As used herein, the term “IVC” or “in-vitro culture” refers to an incubation of fertilized oocytes (zygotes) in the laboratory through the process of cleavage, typically up to the blastocyst stage of development.

As used herein, the term “IVP” or “in-vitro production” refers to a combined process of in-vitro maturation, in-vitro fertilization and in-vitro culture whereby embryos are produced in the laboratory (i.e. IVP=IVM+IVF+IVC).

As used herein, the term “intracytoplasmic sperm injection” refers to a micromanipulation procedure whereby a single spermatozoon is inserted directly into the cytoplasm of the oocyte to achieve fertilization during IVF.

As used herein, the term “blastocyst” refers to a thin-walled hollow structure in early embryonic development that contains a cluster of cells called the inner cell mass from which the embryo arises.

As used herein, the term “inner cell mass” or “trophoblast” refers to a part of the blastocyst that will give rise to the embryo proper, as opposed to the extra-embryonic membranes.

As used herein, the term “implantation” refers to a process whereby the blastocyst stage embryo burrows into the lining of the uterus, or endometrium, to establish a pregnancy.

As used herein, the term “transplantation potential” refers to the capability of an embryo to develop normally to term (birth) following embryonic transplantation, such as by using ART in combination with the present inventions, i.e. a fertilized embryo, a nuclear transplant embryo, and the like.

As used herein, the term “instructions” or “sheet of instructions” as in “instructions for using said kit. In one embodiment for adding follistatin” includes instructions for using the reagents contained in the kit for adding variant (i.e. interfering RNA) and wild type follistatin, such as polypeptides, nucleotides, antibodies, receptor antibodies, and the like.

In one embodiment for “detecting follistatin” such as a diagnostic kit provided for determining transplantation potential, includes instructions for using the reagents contained in the kit for the detection of variant and wild type follistatin nucleotides.

As used herein, “follistatin” refers to any portion of a follistatin molecule, including a nucleic acid and a protein, or portion or fragment thereof, for providing the enhancement of embryonic development as shown herein. Thus, a follistatin may be a molecule from any species of living organism, and refer to any portion of that molecule provided that a showing of enhancement of embryonic development is provided. This enhancement of embryonic development is contemplated to lead to an increase in efficiency for ART of any species, for providing ES cells and any contemplated use of the present inventions related to culturing embryos in follistatin.

In some embodiments, the instructions further comprise the statement of intended use required by the United States (U.S) Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the Federal Food, Drug, and Cosmetic Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the United States Food and Drug Administration.

### DETAILED DESCRIPTION OF THE INVENTION

The present invention provides compositions and methods for increasing the success of assisted reproductive technology (ART). Specifically, the inventions described herein increase the survival rate of manipulated embryos for increasing post implantation numbers of viable offspring. In particular, the present invention provides for compositions and methods for allowing further embryonic development and increasing rates of embryonic maturation, such as increasing cleavage rate, TE numbers, and blastocyte formation of in vitro fertilized and nuclear transfer embryos in media comprising follistatin, thereby providing for increased survival of fertilized and manipulated embryos leading to increased numbers of live offspring from in vitro fertilized and implanted nuclear transfer embryos. Further provided are diagnostic kits for determining transplantation potential.

The inventions provide compositions and methods for increasing the proportion of nuclear transfer embryos that develop to the blastocyst stage of development thereby enhancing the number of trophectoderm cells and the quality of embryos such that an increase in live births from said embryos is contemplated. An increase in blastocyst rate for nuclear transfer embryos would also increase the efficiency of therapeutic cloning and generation of embryonic stem cells for cell therapy.

It was previously demonstrated that follistatin is a marker of egg quality in cattle. The effects of endogenous follistatin on early embryonic development of in vitro fertilized bovine embryos were tested and stimulatory effects on development to the blastocyst stage and number of trophectoderm cells/embryo quality were observed. Given the stimulatory effects on blastocyst development and embryo quality (trophectoderm cells) observed and the observation that the number of trophectoderm cells and quality of nuclear transfer embryos is believed responsible for the less than 5% efficiency of generation of live offspring, the effects of follistatin on development of nuclear transfer embryos to the blastocyst stage and numbers of trophectoderm cells in such embryos was tested.

Results indicate a pronounced stimulatory effect of follistatin on development of nuclear transfer embryos to the blastocyst stage, indicating follistatin treatment results in more transferable embryos. Results also indicate that follistatin treatment significantly enhances embryo quality (number of trophectoderm cells) and results in a greater proportion of live offspring born after transfer of nuclear transfer embryos.

In developing embodiments of the present invention, differences in RNA transcript profiles in oocytes and 2-cell stage bovine embryos associated with oocyte competence were performed. Experimental results indicated that follistatin mRNA is greater in germinal vesicle stage oocytes collected from prepubertal versus adult animals (Patel et al., 2007, Repro. 133:95-106; herein incorporated by reference). Furthermore, follistatin mRNA abundance is greater in early cleaving two cell bovine embryos (collected prior to the maternal zygotic transition and initiation of significant transcription from the embryonic genome) than in their late cleaving counterparts (Patel et al., 2007; herein incorporated by reference). It was contemplated that follistatin had a stimulatory role in early embryonic development. Example 2 demonstrates the effects of exogenous follistatin treatment during the first 72 h of in vitro culture (to 16-cell stage) of bovine embryos on time to first cleavage, development to the blastocyst stage and blastocyst cell allocation (embryo quality). To evaluate the requirement of endogenous follistatin for bovine early embryogenesis, experiments were performed as reported in Example 2 thereby demonstrating the effect of follistatin mRNA knockdown (via microinjection of follistatin siRNA) on time to first cleavage and successful development of bovine embryos to the 8-16 cell and blastocyst stages. Given the robust effects of follistatin treatment on blastocyst development and cell allocation (in favor of trophectoderm) of in vitro fertilized embryos and demonstrated requirement of follistatin for early embryogenesis, it was demonstrated that follistatin treatment enhances the efficiency of nuclear transfer by determining the effects of follistatin supplementation on cell allocation and blastocyst development of nuclear transfer embryos as seen in Example 4.

Certain illustrative embodiments of the invention are described below. The present invention is not limited to these embodiments.

In some embodiments, the present invention provides compositions and methods for identifying and selecting oocytes and embryos with increased levels of endogenous follistatin for nuclear transfer and/or in vitro fertilization procedures. For example, polar bodies that are removed during nuclear transfer cloning procedures are assayed for follistatin. Alternatively, cells of a multi-celled embryo cells as a four-cell, eight-cell or sixteen-cell embryo can be biopsied and analyzed. Assays for follistatin include, but are not limited to, enzyme linked immunosorbent assays (ELISA) (Evans et al., 1998, J. Endo. 156:275-282; herein incorporated by reference). For identifying and selecting post-transfer embryos for increased levels of follistatin for continued culture, one cell from an embryo, for example, is harvested (e.g., embryo biopsy by removing one cell from an embryonic cell mass) and assayed for follistatin levels. When levels of follistatin are increased in either the oocyte or the post-transfer embryo, the nuclear transfer or continued culture, respectively would be carried out. In some embodiments, an additional application of the present invention is the measurement of follistatin in embryo culture media and using it as a guide (positive marker) for selecting embryos to transfer. This has application to human assisted reproductive technologies (ART) (e.g. in vitro fertilization).

In some embodiments, oocytes are harvested and placed in culture media comprising at least 1 ng/ml, at least 5 ng/ml, 10 ng/ml, at least 20 ng/ml, at least 30 ng/ml, at least 40 ng/ml, at least 50 ng/ml, at least 100 ng/ml follistatin, thereby increasing endogenous levels of follistatin in the oocytes prior to nuclear transfer and/or in vitro fertilization. In some embodiments, follistatin is microinjected directly into an oocyte using techniques known by those skilled in the art, thereby increasing levels of endogenous follistatin in said embryo.

In some embodiments, follistatin is used to supplement media generally used to culture embryos. For example, in some embodiments, follistatin is used to supplement potassium simplex oxidized medium (KSOM), a base media for nuclear transfer oocyte and embryo culture. KSOM is typically supplemented with serum (e.g., fetal bovine serum (FBS), human serum, horse serum (HS), etc.) prior to use and may be supplemented with an amino acid cocktail and other components deemed necessary for cell survival. In some embodiments, no serum is present during the time of follistatin supplementation. Exemplary formulations useful in embodiments of the present invention for KSOM can be found in, for example, U.S. Pat. No. 5,541,081, Erbach et al., 1994, Biol. of Repro. 50:1027-1033, and Lawitts and Biggers, 1993, Meth. in Enzym. 225:153-165; all incorporated herein by reference in their entireties. KSOM base media is commercially available from, for example, Millipore Specialty Media. Millipore Specialty Media also provides new KSOM formulations for use as base media for oocyte and embryo culture. However, methods and compositions of the present invention are not limited by the type of culture media used and any type of media and/or supplementation to any media is contemplated, including media systems that are serum free.

KSOM media components typically include, for example, sodium chloride, potassium chloride, potassium hydrophosphate, magnesium sulfate, lactate, glucose, pyruvate, sodium bicarbonate, calcium chloride, glutamine, EDTA, source of mammalian serum (e.g., bovine, fetal bovine, human) and penicillin/streptomycin (e.g., for inhibition of bacterial contamination). PH is typically adjusted to around 7.4. KSOM media is used to culture oocytes and embryos through the blastocyst stage prior to implantation. The KSOM used in the present invention is typically supplemented with BSA (0.3%) or FBS (10%), however follistatin is added during the serum free component of culture. A skilled artisan will understand what is deemed necessary for media supplementation for any particular culture system, not limited to KSOM. In the present application, the KSOM media is further supplemented with follistatin at 1 ng/ml, 10 ng/ml or 100 ng/ml in serum free conditions.

For in vitro fertilization there can be from one to more than one culture and maintenance media used. For example, Gordon in U.S. Pat. No. 5,512,476 (incorporated herein by reference in its entirety) describes the formulation for a culture and fertilization media comprising salts, amino acids, vitamins, pyruvate, glucose, and other components. Alternatively, Gardner and Lane (U.S. Pat. No. 6,838,235; incorporated herein in its entirety) describe the formulations and usage of an 1) oocyte retrieval and handling medium, an 2) oocyte maturation medium, a 3) fertilization medium, 4) several embryonic development media, an 5) embryo transfer media, and 6) a media for cryopreservation. Other formulations for IVF media useful as media in embodiments of the present invention can be found at, for example, U.S. Pat. Nos. 5,627,066, 5,691,194, 6,281,013, and 6,585,982 and in The Handbook of in vitro Fertilization, Eds. Trouson and Gardner, Informa Health Care Publ., 2000, and In vitro Fertilization and Embryo Culture: A Manual of Basic Techniques, Ed. Wolf, Springer Publ., 1988 (all incorporated herein in their entireties). In either a single or a multi-media system, it is contemplated that one or more of the IVF media are supplemented with follistatin at a concentration of, for example, about 1 ng/ml up to about 20 ng/ml.

Methods and compositions for the culture of stem cells are found in, for example, U.S. Pat. Nos. 7,220,584, 7,217,569, 7,148,062, 7,029,913, 7,005,252, 6,200,806, 5,843,780 and 6,328,764; all incorporated herein by reference in their entireties.

In some embodiments, compositions of the present invention comprise nuclear transferred embryos, superovulated embryos, and/or in vitro fertilized embryos cultured in culture media comprising at least 1 ng/ml, at least 5 ng/ml, at least 10 ng/ml, at least 20 ng/ml, at least 30 ng/ml, at least 40 ng/ml, at least 50 ng/ml, at least 100 ng/ml follistatin, thereby increasing survival of the embryos. In some embodiments, the culture media comprises preferably 1.0-20 ng/ml follistatin, more preferably 1-10 ng/ml follistatin. In some embodiments, culture of the nuclear transfer embryos in culture media as previously exemplified provides for differential blastocyst cell allocation. In some embodiments, culture of the nuclear transfer embryos in culture media as previously exemplified further provides for increased numbers of trophectoderm cells in blastocysts in proportion to inner cell mass cells, compared to embryos not grown in follistatin containing media. In some embodiments, follistatin treatment of nuclear transfer embryos increases, for example, their development to blastocyst stage and enhances, for example, blastocyst cell allocation in favor of trophectoderm cells, wherein increased numbers of trophectoderm cells enhances, for example, placentation, and wherein, for example, increased placentation increases the number of live birth offspring from said nuclear transfer embryos grown in follistatin containing media (e.g., at least 1 ng/ml, at least 5 ng/ml, at least 10 ng/ml, at least 20 ng/ml, etc.).

In some embodiments, the present invention provides in vitro fertilization methods that use media supplemented with follistatin. Protocols for performing in vitro fertilization (IVF) can be found at, for example, U.S. Pat. Nos. 4,589,402, 4,725,579 and in The Handbook of in vitro Fertilization, Eds. Trouson and Gardner, Informa Health Care Publ., 2000, and In vitro Fertilization and Embryo Culture: A Manual of Basic Techniques, Ed. Wolf, Springer Publ., 1988; all incorporated herein in their entireties. There are several issues associated with success in performing IVF. Those issues include, but are not limited to, zona pellucida hardening that leads to decrease in sperm penetration, temperature of fertilization and maintenance of eggs, sperm and embryos, pH, the occurrence of volatile organic compounds found in laboratory air that can harm the process, and other environmental factors.

An exemplary protocol for human in vitro fertilization can be divided into several stages; 1) oocyte stimulation, 2) oocyte retrieval, 3) in vitro fertilization, 4) embryo transfer, and 5) post transfer. Basically, ovulation stimulation is induced in a female such that the female starts developing multiple follicles on the ovaries. Gonadotropins such as follicle stimulating hormone or analogues thereof are injected to initiate the developmental process. Spontaneous ovulation is blocked using injections of gonadotropin releasing hormone (GnRH) antagonist that blocks the surge of luteinizing hormone. When follicular maturation is adequate, human chorionic gonadotropin (hCG) is given, and oocytes are retrieved from the female prior to approximately 36 hours after injection of the hCG. Follicles are aspirated from the ovaries and prepared in the laboratory for fertilization with the sperm. The oocytes are incubated with the sperm in culture media as exemplified above for about 18 hours. Fertilization is complete with the observation of two pronuclei in the embryo. However, if fertilization is not realized, for example if sperm count is low, one or more sperm can be injected into the oocyte using intracytoplasmic sperm injections (ICSI). The new embryo(s) is transferred to growth media as exemplified above at, for example, day 3 prior to the blastocyst stage, at 5 day (blastocyst stage), and sometimes embryos are transferred at the 6-8 cell stage. In some embodiments, the growth media comprises follistatin as described above. In some embodiments, the measurement of follistatin in culture media is used to predict which cultures of embryos to select for transfer contemplated to have the greatest potential to reach the blastocyst stage and beyond. Predicting embryos with greatest potential includes, but is not limited to, those embryos in culture where increased amounts of follistatin in the media is present, in comparison to embryo cultures with lesser amount of follistatin present.

Further, embryos in culture may provide biopsies for diagnostic tests, including but not limited to genetic tests, protein tests, and protein expression, such as preimplantation genetic diagnosis, genetic compatibility tests (i.e. blood type (ABO factors), Rhesus factors (Rh factors + or −) tests, Major Histocompatibility Complex (MHC) molecules, Types I and II,) tests, etc., and mRNA expression diagnosis. Such that, in another embodiment, a diagnostic test is contemplated for the relative amount of follistatin mRNA, protein, and the like, in the embryo biopsy for estimating transplantation potential. In a further embodiment, a diagnostic tests comprises a test for follistatin protein, follistatin mRNA, and diagnostic tests including but not limited to those listed herein, see, supra.

Early cleavage of human embryos to the two-cell stage after intracytoplasmic sperm injection is an indicator of embryo viability, (for example, see, Sakkas, et al., 1998 Human Reproduction, 13:182-187; herein incorporated by reference).

Support for such diagnostic use in animals and primates, including humans, is provided in Patel, et al., Reproduction (2007) 133 95-106; herein incorporated by reference in its entirety, wherein microarry experiments indicated a positive association between time of first cleavage (oocyte competence) and follistatin mRNA abundance. Follistatin, BB, and BA subunits of inhibin/activin mRNAs were temporally regulated during early bovine embryogenesis and peaked at the 16-cell stage. Collectively, results demonstrate a positive association of follistatin mRNA abundance with oocyte competence in two distinct models and dynamic regulation of follistatin, BB, and subunit mRNAs in early embryos after initiation of transcription from the embryonic genome. The differences in timing of first cleavage are contemplated to be reflective of inherent differences in transcriptome composition (maternally derived transcripts) between early and late cleaving embryos at the two-cell stage.

For ART, embryos, after grading by an embryologist, are transferred into the female uterus. Oftentimes, multiple embryos are transferred at one time thereby improving the change of implantation and pregnancy, albeit this can also results in multiple births if all embryos remain viable and develop.

After embryo transfer, the female typically receives progesterone shots so that the uterus lining thickens and remains thick and suitable for implantation. Approximately two weeks post-implantation the female is checked for pregnancy.

Protocols change for different mammalian species; the protocol listed above is exemplary of a human IVF protocol. However, the present application is not limited to human IVF only, and IVF for other non-human animals is equally amendable with the compositions and methods of the present application. For example, protocol for bovine IVF can be found at, for example, Beyhan et al., 2007, Dev. Biol. 305:637-649, incorporated herein by reference in its entirety.

In some embodiments, the present invention provides nuclear transfer methods that utilize the media of the present invention. Methods for nuclear transfer cloning can be found at, for example, U.S. Pat. Nos. 6,147,276, 6, 252,133, 6,525,243, 2007/0033664, and 2007/0033665 and in Beyhan et al., 2007; all incorporated herein in their entireties. Nuclear transfer techniques fall into two categories: 1) transfer of a donor nucleus (or cell containing a donor nucleus) to a matured metaphase II oocyte which has had its chromosomal DNA removed (e.g., polar body removed) and 2) transfer of a donor nucleus to a fertilized one cell zygote which has had both pronuclei removed. In ungulates the former procedure has become the method of choice. Transfer of the donor nucleus into the oocyte cytoplasm is generally achieved by inducing cell fusion. In ungulates fusion is induced by application of a DC electrical pulse across the contact/fusion plane of the couplet. The same pulse which induces cell fusion can also activate the recipient oocyte. In developing embodiments of the present invention ionomycin was used to induce activation of the recipient oocyte and an electrical pulse was used to induce fusion. Following embryo reconstruction further development is dependent on a large number of factors including the ability of the nucleus to direct development (i.e. totipotency), developmental competence of the recipient cytoplast (i.e. oocyte maturation), oocyte activation, embryo culture (reviewed in Campbell and Wilmut, 1994, in: Vth World Congress on Genetics as Applied to Livestock 20 180-187; incorporated herein by reference in its entirety). As such, the present invention provides methods for predicting or enhancing developmental competence of the recipient cytoplast.

Three methods which have been used to induce fusion are: (1) exposure of cells to fusion-promoting chemicals, such as polyethylene glycol; (2) the use of inactivated virus, such as Sendai virus; and (3) the use of electrical stimulation. Exposure of cells to fusion-promoting chemicals such as polyethylene glycol or other glycols is a routine procedure for the fusion of somatic cells, but it has not been widely used with embryos. As polyethylene glycol is toxic it is necessary to expose the cells for a minimum period and the need to be able to remove the chemical quickly may necessitate the removal of the zona pellucida (Kanka et al., 1991, Mol. Reprod. Dev. 29:110-116; herein incorporated by reference). In experiments with mouse embryos, inactivated Sendai virus provides an efficient means for the fusion of cells from cleavage-stage embryos (Graham, 1969, Wistar Inst. Symp. Monogr. 9:19; herein incorporated by reference), with the additional experimental advantage that activation is not induced. In ungulates, fusion is commonly achieved by the same electrical stimulation that is used to induce parthenogenetic activation (Willadsen, 1986, Nature 320:63-65); Prather et al., 1987, Biol. Reprod. 37 859-866; all of which are herein incorporated by reference). In these species, Sendai virus induces fusion in a proportion of cases, but is not sufficiently reliable for routine application (Willadsen, 1986, Nature 320:63-65; herein incorporated by reference).

While cell-cell fusion is a preferred method of effecting nuclear transfer, it is not the only method that can be used. Other suitable techniques include microinjection (Ritchie and Campbell, J. Reproduction and Fertility Abstract Series No. 15, p 60; herein incorporated by reference).

Subsequently, the fused reconstructed embryo is maintained without being activated so that the donor nucleus is exposed to the recipient cytoplasm for a period of time sufficient to allow the reconstructed embryo to become capable, eventually, of giving rise to a live birth (preferably of a fertile offspring).

The optimum period of time before activation varies from species to species. For example, for cattle, a period of from 6 to 20 hours is appropriate (e.g., activation with ionomycin for 4 minutes and culture in presence of cytocholasin B and cycloheximide for 6 h). The time period should probably not be less than that which will allow chromosome formation and it should not be so long either that the couplet activates spontaneously or, in extreme cases that it dies. When it is time for activation, any conventional or other suitable activation protocol is used. Recent experiments have shown that the requirements for parthenogenetic activation are more complicated. It had been assumed that activation is an all-or-none phenomenon and that the large number of treatments able to induce formation of a pronucleus were all causing “activation”. However, exposure of rabbit oocytes to repeated electrical pulses revealed that only selection of an appropriate series of pulses and control of the Ca²⁺ was able to promote development of diploidized oocytes to mid-gestation (Ozil, 1990, Development 109:117-127; herein incorporated by reference). During fertilization there are repeated, transient increases in intracellular calcium concentration (Cutbertson & Cobbold, 1985, Nature 316:541-542; herein incorporated by reference) and electrical pulses are believed to cause analogous increases in calcium concentration. There is evidence that the pattern of calcium transients varies with species and it can be anticipated that the optimal pattern of electrical pulses will vary in a similar manner. For example, the interval between pulses in the rabbit is approximately 4 minutes (Ozil, 1990, Development 109:117-127; herein incorporated by reference), and in the mouse 10 to 20 minutes (Cutbertson & Cobbold, 1985, Nature 316:541-542; herein incorporated by reference), while there are preliminary observations in the cow that the interval is approximately 20 to 30 minutes (Robl et al., 1992, in: Symposium on Cloning Mammals by Nuclear Transplantation (Seidel ed.), Colorado State University, 24-27; herein incorporated by reference).

In most published experiments activation was induced with a single electrical pulse, but new observations suggest that the proportion of reconstituted embryos that develop is increased by exposure to several pulses (Collas & Robl, 1990, Biol. Reprod. 43:877-884; herein incorporated by reference). In any individual case, a skilled artisan will recognize that routine adjustments are made to optimize the number of pulses, the field strength and duration of the pulses and the calcium concentration of the medium. Other factors also contribute to controlling the time allowed for reprogramming, such as culture in the presence of DMAp, cytocholasin B, and the like.

At the blastocyst stage, the embryo is screened for suitability for development to term. Typically, this is done where the embryo is transgenic and screening and selection for stable integrants has been carried out. Screening for non-transgenic genetic markers may also be carried out at this stage. However, screening of donors at an earlier stage, as described in the present invention, are preferred.

After screening, the blastocyst embryo is allowed to develop to term. This will generally be in vivo. If development up to blastocyst has taken place in vitro, then transfer into the final recipient animal takes place at this stage. If blastocyst development has taken place in vivo, although in principle the blastocyst can be allowed to develop to term in the pre-blastocyst host, in practice the blastocyst will usually be removed from the (temporary) pre-blastocyst recipient and, after dissection from the protective medium, will be transferred to the (permanent) post-blastocyst recipient.

### I. The Use of Follistatin in the Present Inventions.

In some embodiments, the present invention provides methods for the propagation of non-human animals, such as livestock animals. In some embodiments, the propagation of non-human animals comprising, for example, in vitro fertilization and nuclear transfer cloning of embryos. In some embodiments, the embryos are placed in culture media comprising follistatin, wherein said follistatin level is at least 1 ng/ml, at least 5 ng/ml, at least 10 ng/ml, at least 20 ng/ml, at least 30 ng/ml, at least 40 ng/ml, at least 50 ng/ml, at least 100 ng/ml follistatin. In some embodiments, maintenance and/or growth of in vitro fertilization and nuclear transfer embryos in follistatin comprising media provides for an increased survival of embryos in vitro and in vivo (e.g., post-implantation) that, for example, develop into viable offspring. In some embodiments, the present invention provides methods for generation of transgenic animals. In some embodiments, newly fertilized embryos used for transgenic gene transfer are maintained/grown in vitro in follistatin comprising media, wherein said follistatin level is at least 1 ng/ml, at least 5 ng/ml, at least 10 ng/ml, at least 20 ng/ml, at least 30 ng/ml, at least 40 ng/ml, at least 50 ng/ml, at least 100 ng/ml follistatin. In some embodiments, maintenance/growth of transgenic embryos in follistatin comprising media provides for an increased survival of embryos in vitro and in vivo (e.g., post-implantation) that, for example, develop into viable offspring.

In humans, the causes of infertility are complex. A large proportion of cases of infertility are attributed, at least in part, to dysfunction of the female reproductive system [Centers, for, Disease, Control, and, Prevention. Assisted reproductive technology success rates: national summary and fertility clinic reports. In; 2001: www.cdc.gov/reproductivehealth/art.htm; herein incorporated by reference] and specifically to a reduction in oocyte quality, particularly in the case of women of advanced reproductive age [Fitzgerald, et al., Yale J Biol Med 1998; 71: 367-381; herein incorporated by reference]. While the past decade has generally brought advancements in efficacy and safety of ART, there is still much room for improvement. The most recent statistics available from the CDC indicate an overall average success rate of 29% live births from a single ART cycle, but success rate is diminished with increased maternal age [Centers, for, Disease, Control, and, Prevention. Assisted reproductive technology success rates: national summary and fertility clinic reports. In; 2001: www.cdc.gov/reproductivehealth/art.htm; herein incorporated by reference, Krey, et al., Ann NY Acad Sci 2001; 943: 26-33; herein incorporated by reference]. The tangible impact of increased maternal age on ART success cannot be dismissed, given clear societal trends for delaying childbirth until later in life [Fitzgerald, et al., Yale J Biol Med 1998; 71: 367-381; herein incorporated by reference, te Velde, [editorial]. Maturitas 1998; 30: 103-104; herein incorporated by reference].

Furthermore, while the incidence of triplet and higher order pregnancies resulting from ART has decreased since 1997 [Rebar, et al., N Engl J Med 2004; 350: 1603-1604; herein incorporated by reference], the incidence of singleton pregnancies has not increased [Jain, et al., N Engl J Med 2004; 350: 1639-1645; herein incorporated by reference]. Multiple births resulted in $640 million in initial hospital costs in 2000, due to increased infant mortality and morbidity [Hogue, Obstetrics and Gynecology, 2002, 100:1017-1019; herein incorporated by reference].

Births from human ART account for greater than 30% of all twin births and greater than 40% of triplet and higher births [Hogue, Obstetrics and Gynecology, 2002, 100: 1017-1019; herein incorporated by reference]. Thus there is a need for much improvements in the efficacy and safety of ART whose success if limited by a dearth of fundamental knowledge of the intracellular and intercellular mechanisms and mediators of oocyte competence and a lack of measurable, objective characteristics reflective of the quality or competence of individual human oocytes at time of collection and appropriate therapeutic approaches to enhance oocyte quality and thus embryonic development following fertilization. Elucidation of such foundational information may ultimately revolutionize ART and form the basis for diagnostic approaches to increase oocyte competence and pregnancy outcomes and (or) diagnostic approaches to enhance embryo development and quality potentially allow for fertilization and (or) subsequent transfer of only those embryos derived from the best quality oocytes collected and hence those with the greatest chance for pregnancy success.

Thus the inventors contemplate the use of the present inventions to meet the unmet need for enhancing embryonic development in vitro. The enhancement of embryonic development of the present inventions is contemplated to result in the increase of live births for overcoming infertility, in particular for overcoming infertility without the unwanted side effects, as described herein, such as unwanted multiple births.

In some embodiments, compositions and methods of the present inventions provide for the generation and growth of stem cells obtained from noncloned embryos and cloned embryos, such as for treating human disease and injury, for use in overcoming genetic incompatibility, provided specific cell types for transplantation, and “embryo-friendly” derivation methods for providing (Stem Cells Vol. 24 No. 10 Oct. 2006, pp. 2162-2169; herein incorporated by reference). These potential uses are based upon the observation that these cells can, under appropriate conditions in vitro or in vivo, differentiate into most, if not all, cell types of the adult human body.

The path to successful hES (human embryonic stem cell) therapies appear straightforward, making the desired cell type from hES cells, such as neurons to treat neurodegenerative disease or pancreatic β-cells to treat type I diabetes, and then transfer these cells to the desired site. However there are many difficulties in producing such cell, such that Embryonic stem (ES)-derived cells of the present inventions would also provide a source of differenctiated cells, growth factors, and signaling molecules for further use in stem cell therapies.

In some embodiments, compositions and methods of the present inventions provide for the using and maintenance noncloned embryos and cells from those embryos useful for therapeutic purposes including, but not limited to, the growth of stem cells. In some embodiments, compositions and methods of the present inventions provide for the cloning and maintenance of cells useful for therapeutic purposes including, but not limited to, the growth of stem cells. For example, nuclear transfer embryos comprising nuclear material from a somatic cell can be maintained/grown in follistatin comprising culture media. Growth of such embryos, due to culture in follistatin comprising media, will exhibit increased numbers of embryos reaching blastocyst stage, thereby, increasing the numbers of cells produced, including, but not limited to, the number of stem cells produced. As such, increased numbers of stem cells are produced per nuclear transfer embryo when applying compositions and methods of the present invention and more blastocyst stage embryos per recipient cytoplast utilized are available for generation of stem cells. Stem cells produced using the compositions and methods of the present invention find utility in, for example, therapeutics, drug discovery, and applied research.

Somatic cell nuclear transfer (SCNT) is a laboratory technique for creating an ovum with a donor nucleus. It is used, for example, in embryonic stem cell research, or in regenerative medicine where it is sometimes referred to “therapeutic cloning.” It can also be used as the first step in the process of reproductive cloning. In SCNT the nucleus of a somatic cell is removed and the rest of the cell discarded. At the same time, the nucleus of an egg cell is removed. The nucleus of the somatic cell is then inserted into the enucleated egg cell, typically through a microscopic glass tube. After being inserted into the egg, the somatic cell nucleus is reprogrammed by the host cell. The egg, now containing the nucleus of a somatic cell, is stimulated with an electrical shock and begins to divide in culture. After many mitotic divisions in culture, this single cell forms a blastocyst with almost identical DNA to the original organism. Inner cell mass are subsequently removed, and stem cells grown out and harvested and used in, for example, research, and/or to generate tissues and cells (therapeutic cloning).

Methods for SCNT and culture of embryos and stem cells generated can be found at, for example, Wilmut et al., 2002, Nature 419:583-586; Alberio et al., 2006, Repro. 132:709-720; Vanikar et al., 2007, Transp. Proc. 39:658-661; Wakayama et al., 2001, Science, 292:740-743; all incorporated herein by reference in their entireties. Compositions and methods of the present invention provide for developing stem cell generating embryos that are healthier and thereby generate more stem cells.

In some embodiments, the media of the present invention also find use for the development and culture of embryos used to make stem cell lines, such as primate stem cell lines. Methods for obtaining pluripotent cells from species in these animal orders, including monkeys, mice, rats, pigs, cattle and sheep have been previously described. See, e.g., U.S. Pat. Nos. 5,453,357; 5,523,226; 5,589,376; 5,340,740; and 5,166,065 (all of which are specifically incorporated herein by reference); as well as, Evans, et al., Theriogenology 33(1):125-128, 1990; Evans, et al., Theriogenology 33(1):125-128, 1990; Notarianni, et al., J. Reprod. Fertil. 41(Suppl.):51-56, 1990; Giles, et al., Mol. Reprod. Dev. 36:130-138, 1993; Graves, et al., Mol. Reprod. Dev. 36:424-433, 1993; Sukoyan, et al., Mol. Reprod. Dev. 33:418-431, 1992; Sukoyan, et al., Mol. Reprod. Dev. 36:148-158, 1993; Iannaccone, et al., Dev. Biol. 163:288-292, 1994; Evans & Kaufman, Nature 292:154-156, 1981; Martin, Proc Natl Acad Sci USA 78:7634-7638, 1981; Doetschman et al. Dev Biol 127:224-227, 1988); Giles et al. Mol Reprod Dev 36:130-138, 1993; Graves & Moreadith, Mol Reprod Dev 36:424-433, 1993 and Bradley, et al., Nature 309:255-256, 1984; all of which are herein incorporated by reference.

Primate embryonic stem cells may be preferably obtained by the methods disclosed in U.S. Pat. Nos. 5,843,780 and 6,200,806, each of which is incorporated herein by reference. Primate (including human) stem cells may also be obtained from commercial sources such as WiCell, Madison, Wis. A preferable medium for isolation of embryonic stem cells is “ES medium.” ES medium consists of 80% Dulbecco's modified Eagle's medium (DMEM; no pyruvate, high glucose formulation, Gibco BRL), with 20% fetal bovine serum (FBS; Hyclone), 0.1 mM P-mercaptoethanol (Sigma), 1% non-essential amino acid stock (Gibco BRL). Preferably, fetal bovine serum batches are compared by testing clonal plating efficiency of a low passage mouse ES cell line (ESⱼₜ₃), a cell line developed just for the purpose of this test. FBS batches must be compared because it has been found that batches vary dramatically in their ability to support embryonic cell growth, but any other method of assaying the competence of FBS batches for support of embryonic cells will work as an alternative.

Primate ES cells are isolated on a confluent layer of murine embryonic fibroblast in the presence of ES cell medium. Embryonic fibroblasts are preferably obtained from 12 day old fetuses from outbred CF1 mice (SASCO), but other strains may be used as an alternative. Tissue culture dishes are preferably treated with 0.1% gelatin (type I; Sigma). Recovery of rhesus monkey embryos has been demonstrated, with recovery of an average 0.4 to 0.6 viable embryos per rhesus monkey per month, Seshagiri et al. Am J Primatol 29:81-91, 1993; herein incorporated by reference. Embryo collection from marmoset monkey is also well documented (Thomson et al. “Non-surgical uterine stage preimplantation embryo collection from the common marmoset,” J Med Primatol, 23:333-336 (1994); herein incorporated by reference). Here, the zona pellucida is removed from blastocysts by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes.

After two further washes in DMEM, lysed trophectoderm cells are removed from the intact inner cell mass (ICM) by gentle pipetting, and the ICM plated on mouse inactivated (3000 rads gamma irradiation) embryonic fibroblasts. After 7-21 days, ICM-derived masses are removed from endoderm outgrowths with a micropipette with direct observation under a stereo-microscope, exposed to 0.05% Trypsin-EDTA (Gibco) supplemented with 1% chicken serum for 3-5 minutes and gently dissociated by gentle pipetting through a flame polished micropipette.

Dissociated cells would be replated on embryonic feeder layers in fresh ES medium, and observed for colony formation. Colonies demonstrating ES-like morphology would then be individually selected, and split again as described above. ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (without calcium or magnesium and with 2 mM EDTA) every 1-2 weeks as the cultures become dense. Early passage cells would be frozen then stored in liquid nitrogen.

In one embodiment, the present invention provides compositions for increasing embryo quality. In some embodiments, the present invention provides media that maintains and/or increases embryo quality after the embryos have been frozen and re-thawed. A problem in storage of embryos for later use (e.g., for later use in in vitro fertilization or nuclear transfer) is that embryos do not tolerate freezing well and many embryos die as a result of the storage conditions (e.g., embryos in media and liquid nitrogen freezing). The present invention provides media for embryo storage such that embryos, when stored (e.g., frozen) in media of the present invention are more tolerant to the freezing conditions. As such, the present invention provides compositions for increasing an embryo's tolerance to freezing thereby increasing the overall quality of the embryo.

In further embodiments, the inventors contemplate additional measures of determining embryo quality, including cryotolerance, gene expression markers, such as those described herein, blastomere apoptosis, etc. As such, the inventors contemplate the use of adding additional molecules to medium comprising follistatin, including but not limited to any agonists or antagonists of action of TGF beta superfamily members providing these molecules enhance the development and/or quality of embryonic development and/or stem cell growth.

### II. Kits.

The present invention also provides kits for determining whether a cell or tissue is expressing a follistatin gene. The present invention also provides kits for adding follistatin to a cell medium. The kits are produced in a variety of ways. In some embodiments, the kits contain at least one reagent for specifically adding a follistatin allele or protein. In one embodiment, the kit contains an oligonucleotide reagent for detecting an expressed follistatin cDNA. In some embodiments, the kits contain at least one reagent for specifically adding an additional compound.

In some embodiments, the kits include ancillary reagents such as buffering agents, nucleic acid stabilizing reagents, protein stabilizing reagents, and signal producing systems (e.g., florescence generating systems as Fret systems). The kit may be packages in any suitable manner, typically with the elements in a single container or various containers as necessary along with a sheet of instructions for carrying out the test. In some embodiments, the kits also preferably include a negative control such as a siRNA of the present inventions.

### EXPERIMENTAL

The following examples are provided in order to demonstrate and further illustrate certain preferred embodiments and aspects of the present invention and are not to be construed as limiting the scope thereof.

### Example 1

### Follistatin Supplementation of IVF Embryos

The effects of follistatin treatment on in vitro produced bovine embryos during the initial 72 h (hour) post fertilization on time to first cleavage (at least a 2 cell embryo), development to the blastocyst stage (day 7, d7) and blastocyst cell allocation (quality) was determined.

Cumulus-oocyte complexes (COCs) were harvested from ovaries obtained from a local abattoir (slaughterhouse), matured and fertilized in vitro. In vitro maturation of bovine oocytes, and in vitro fertilization and culture of embryos to the blastocyst stage were conducted as reported previously (Bettegowda et al., 2006, Mol. Repro. Dev. 73:267-278; herein incorporated by reference). After 20 h of co-incubation with spermatozoa, presumptive zygotes were stripped of cumulus cells and cultured in KSOM medium supplemented with 0.3% BSA containing 0, 1, 10 or 100 ng/ml Recombinant Human Follistatin (R&D Systems, Minneapolis, Minn., United States) referred to as “follistatin” in these Examples, respectively, (n=25 presumptive zygotes per treatment, n=6 replicates).

The proportion of embryos reaching the two-cell stage within 30 h (early cleaving), 30-36 h (late cleaving) and within 48 h post fertilization (total cleavage rate) was determined. Embryos at the 8-16-cell stage were separated 72 h after fertilization and cultured in fresh potassium-enriched simplex optimized medium (KSOM medium) supplemented with 0.3% BSA and 10% FBS until day 7. The number of embryos reaching the blastocyst stage at d 7 post fertilization was recorded and numbers of inner cell mass (ICM) and trophectoderm (TE) cells determined by differential staining.

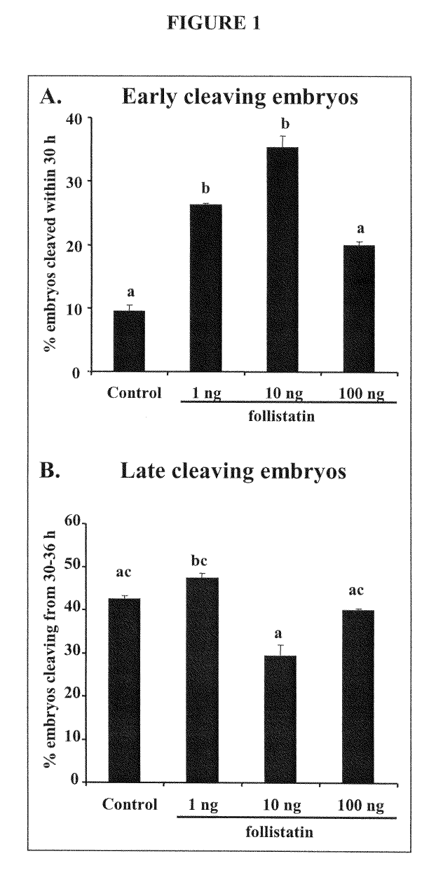

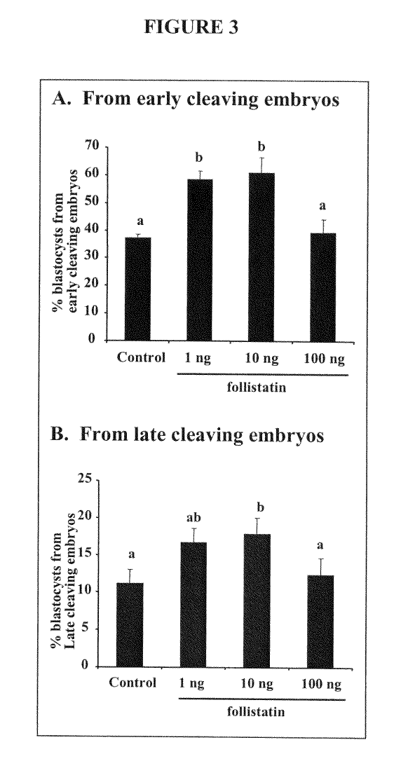

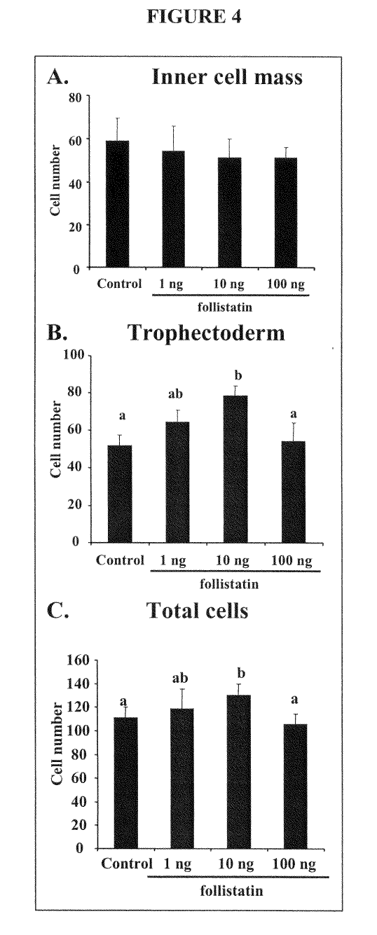

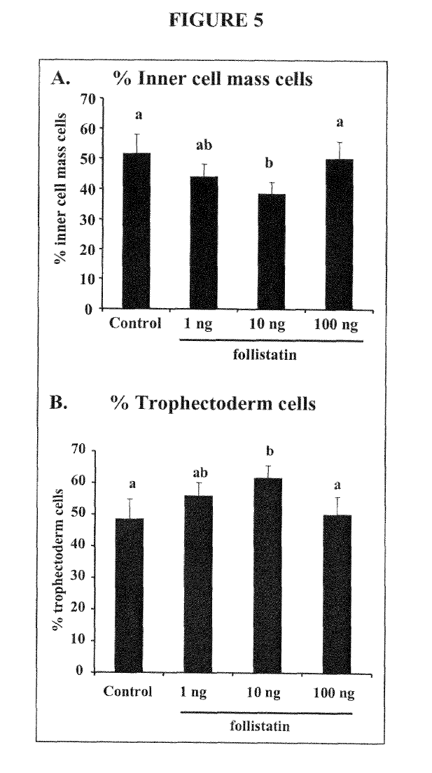

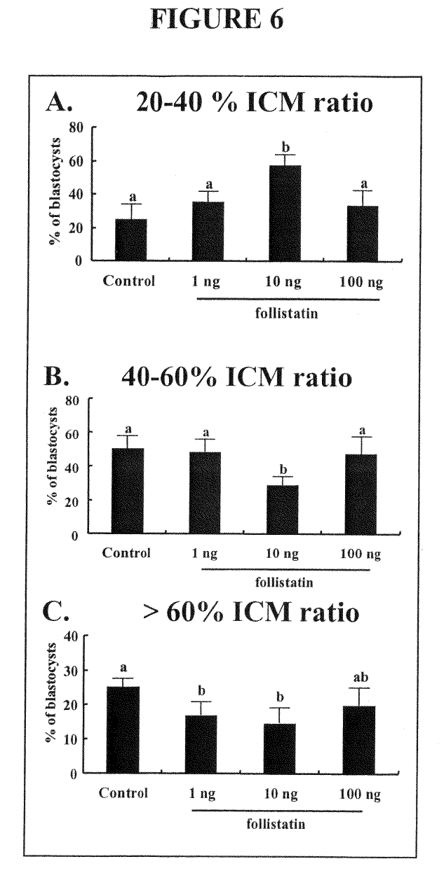

Follistatin treatment did not increase the rate of total cleavage or affect proportion of late cleaving embryos when compared to control. However, supplementation with 1 and 10, but not 100 ng/ml follistatin increased the proportion of early cleaving embryos and development to the blastocyst stage relative to controls (FIGS. 1 & 2, respectively). When early and late cleaving embryos were separated and cultured separately, follistatin treatment (1 and 10 ng/ml) of early cleaving embryos increased (P<0.05) the rate of development to blastocyst stage and a stimulatory effect of 10 ng/ml follistatin on day 7 blastocyst rate for late cleaving embryos was also observed relative to untreated controls (FIG. 3). Treatment with 10 ng/ml follistatin increased (P<0.05) total cell numbers and trophectoderm cell numbers, but did not affect numbers of inner cell mass cells relative to untreated embryos (FIG. 4). Treatment with 10 ng/ml follistatin also increased (P<0.05) proportion of trophectoderm cells and reduced the ICM/total cell ratio in day 7 blastocysts relative to controls (FIG. 5). Furthermore, treatment with 10 ng/ml follistatin increased the proportion of day 7 blastocysts with a 20-40% ratio of inner cell mass to total cells (similar to in vivo produced embryos) and decreased the proportion of day 7 blastocysts with 40-60% and >60% ICM/total cell ratios (P<0.05) (FIG. 6). Embryos with ICM ratio of 20-40% are presumed to be higher quality as >80% of in vivo-derived blastocysts were classified into this category in previous studies (Koo et al., 2002, Biol. Repro. 67:487-492; herein incorporated by reference).

### Example 2

### siRNA Mediated Knockdown of Follistatin mRNA and its Effect on Development of IVF Embryos

To determine the need for endogenous follistatin for early embryonic development, the effect of follistatin RNA knockdown (e.g., via microinjection of follistatin siRNA) on time to first cleavage and successful development of in vitro fertilized bovine embryos to the 8-16 cell and blastocyst stages were determined. After 20 h of co-incubation with spermatozoa, presumptive zygotes were stripped of cumulus cells and cultured in KSOM medium supplemented with 0.3% BSA and microinjected with follistatin siRNA (25 μM). Presumptive zygotes injected with similar concentration of negative control siRNA (Ambion Universal Control #1) or water (sham microinjection and uninjected 1 embryos all served as controls).

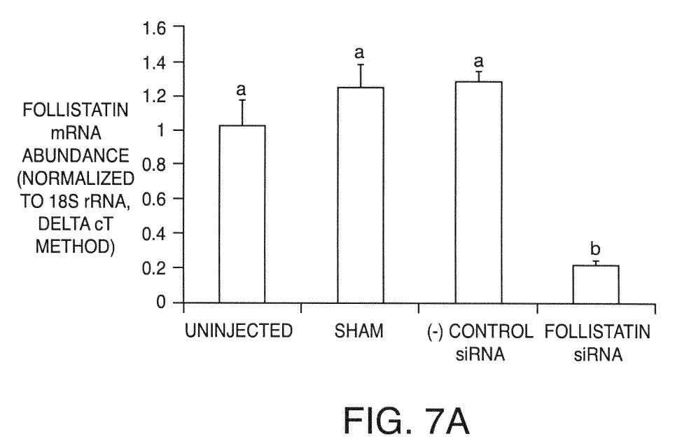

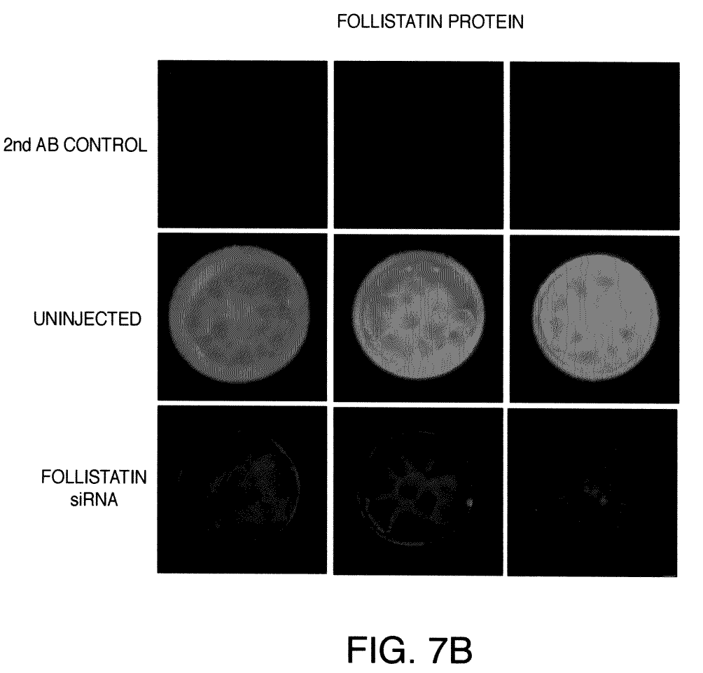

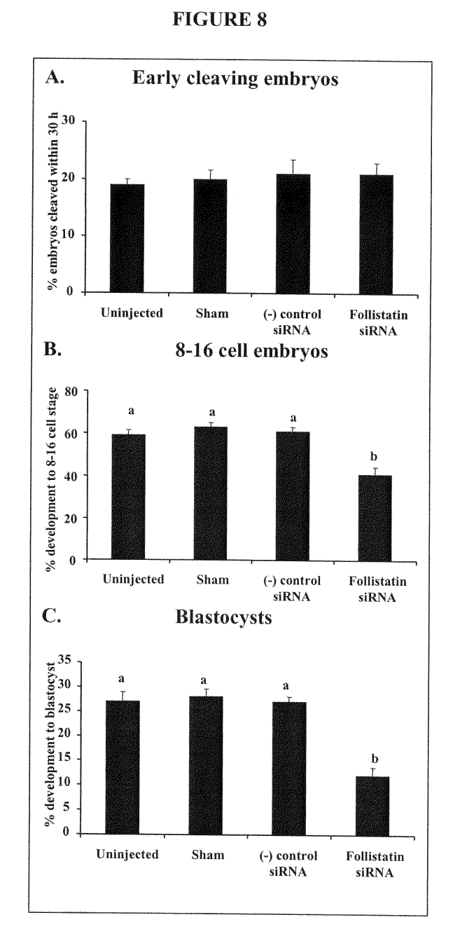

Follistatin siRNA injection (25 μM) into presumptive zygotes decreased amounts of follistatin mRNA at the 4-cell stage (FIG. 7) by approximately 80% relative to uninjected, sham injected and negative control siRNA injected embryos (P<0.05), and dramatically reduced follistatin protein in 16-cell embryos (determined by immunofluorescence) relative to uninjected controls demonstrating efficacy and specificity of follistatin siRNA in reducing follistatin mRNA and protein abundance in early bovine embryos (FIG. 7). Microinjection of follistatin siRNA into presumptive zygotes did not influence proportion of embryos cleaving early (within 30 h) but reduced proportion of embryos developing to the 8-16 cell and blastocyst relative to uninjected, sham injected and negative control siRNA injected embryos and proportion of day 7 blastocysts (FIG. 8). As such, experimental results as found in FIGS. 1-8 provide evidence for a role of follistatin in early embryogenesis.

### Example 3

### Follistatin Supplementation Effects on the Development of Nuclear Transfer Embryos

To demonstrate that follistatin treatment enhances the efficiency of nuclear transfer, the effects of follistatin supplementation on cell allocation and blastocyst development of nuclear transfer embryos was determined. The generation of nuclear transfer embryos was conducted as described by Beyhan et al., 2007, Dev. Biol. 305:637-649; herein incorporated by reference. After nuclear transfer, activation and fusion, embryos were cultured as described in Example 2 (n=4 replicates of 25 embryos per treatment) in the presence of 0, 1, 10 and 100 ng/ml of follistatin. Untreated parthenogenetic embryos were included as quality control for oocytes and the activation procedure. The numbers of trophectoderm (TE) versus inner cell mass (ICM) cells and total cell numbers were determined by cell counts on differentially stained embryos (Machaty et al., 1998, Biol. Repro. 59:451-455; herein incorporated by reference).

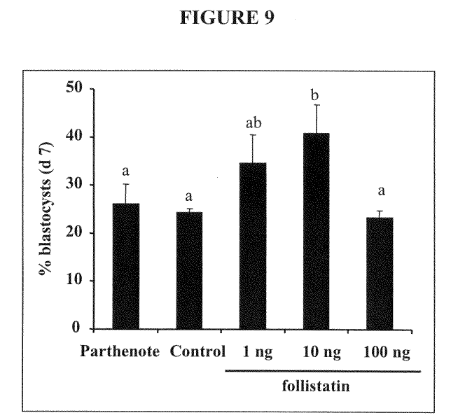

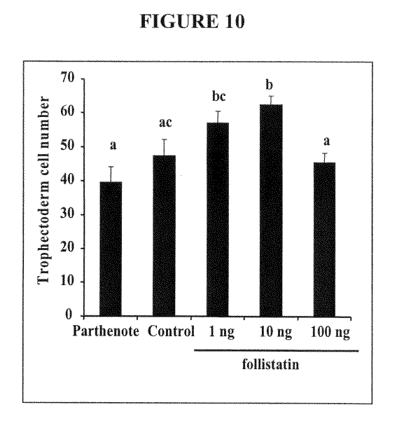

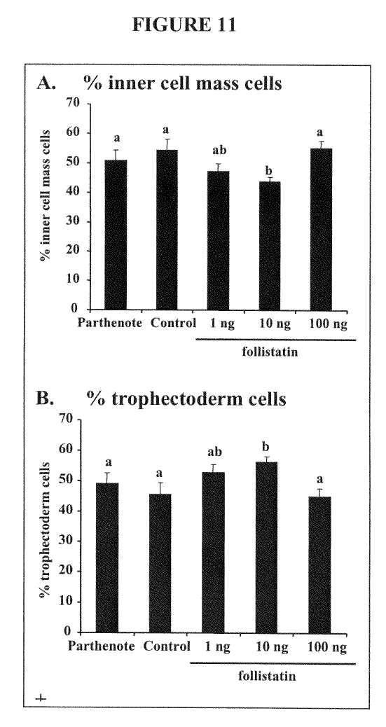

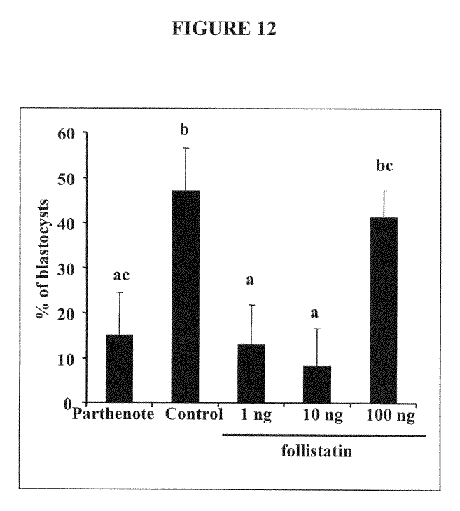

Due to defects in placentation and blastocyst cell allocation characteristic of nuclear transfer embryos (presumably leading to high rates of pregnancy loss), this experiment was conducted to determine the effects of follistatin supplementation on development of nuclear transfer embryos to the blastocyst stage and on blastocyst cell allocation. Treatment with 10 ng/ml follistatin significantly increased the proportion of nuclear transfer embryos developing to the blastocyst stage relative to untreated nuclear transfer embryos (P<0.05) (FIG. 9). While not significantly affecting numbers of ICM and total cells, treatment with 10 ng/ml follistatin significantly increased numbers of trophectoderm cells and proportion of trophectoderm cells to total cells and decreased the proportion of inner cell mass cells to total cells relative to untreated nuclear transfer embryos (P<0.05) (FIG. 10). No significant effect of other doses of follistatin was observed for the above endpoints. However, treatment with 1 and 10 ng/ml follistatin reduced the proportion of day 7 nuclear transfer blastocysts with an ICM ratio of >60% relative to untreated nuclear transfer blastocysts (FIG. 11). No significant effect of follistatin treatment on proportions of day 7 nuclear transfer blastocysts with ICM ratio of 20-40% or 40-60% was observed (FIG. 12).

Collectively, the results of the above studies demonstrate that exogenous follistatin treatment during the early stages of in vitro bovine embryo development enhances time to first cleavage, development to the blastocyst stage and cell allocation in favor of increased trophectoderm cells, thereby supporting a functional requirement of follistatin for early embryonic development. The results also indicate that follistatin treatment can be used to increase development of nuclear transfer embryos to the blastocyst stage and enhance blastocyst cell allocation in favor of trophectoderm cells for enhancing placentation and ultimately birth of live offspring.

### Example 4

### This Example Demonstrates an Exemplary Follistatin Specific on Embryonic Development

Specifically, a negative effect of siRNA mediated knockdown of follistatin mRNA was demonstrated using in vitro fertilized embryos and capability of exogenous follistatin to rescue embryonic development.

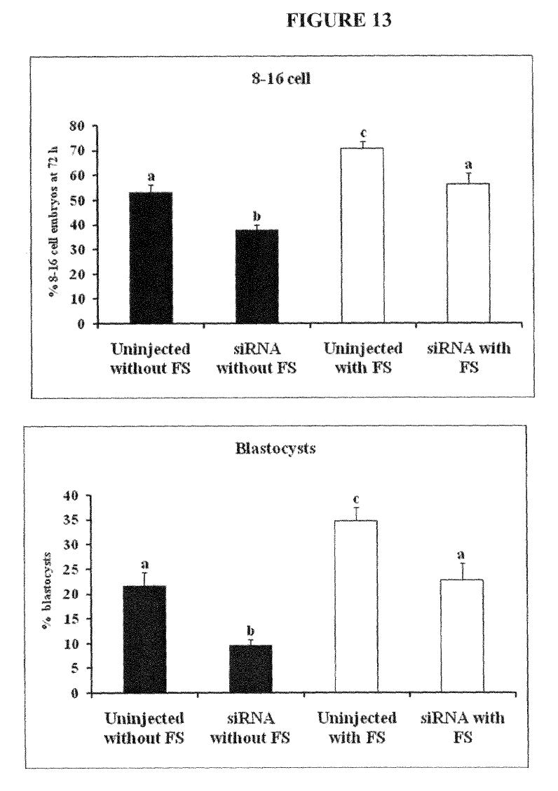

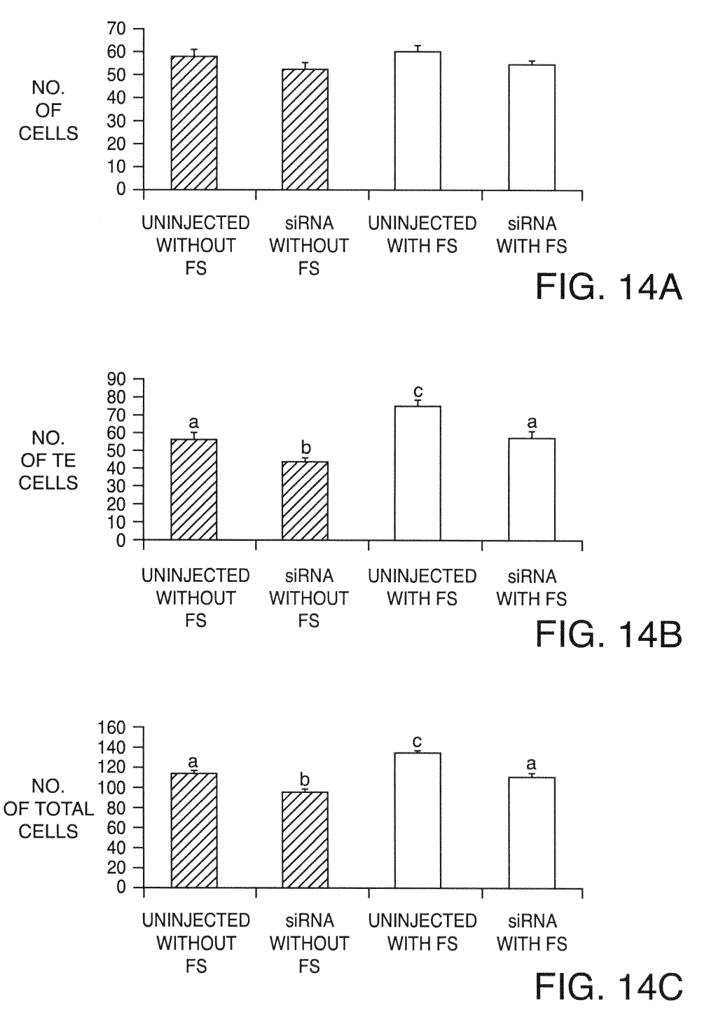

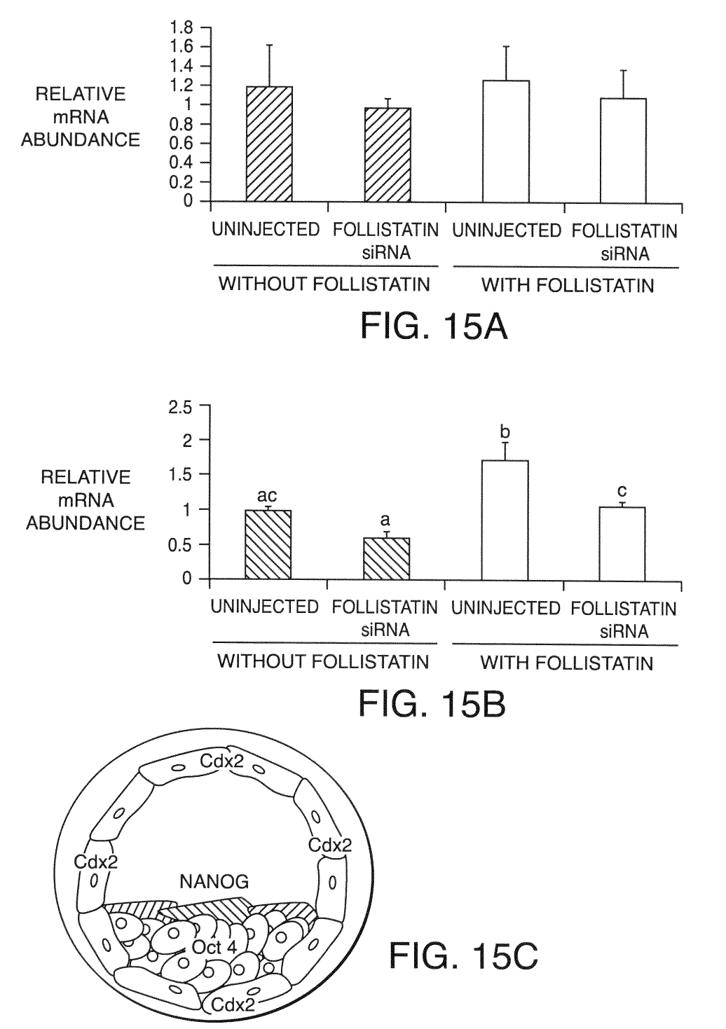

Experiments such as those described in Example-2 (FIG. 8) were initiated for inhibiting endogenous follistatin expression and production. As an additional experimental condition endogenous follistatin was lowered using the siRNA construct in the presence of exogenous follistatin at 10 ng/ml in the culture medium. Exogenous follistatin. Rescued the percent 8-16 cell embryos at 72 hr, and recovered the percent in blastocyte stage (FIG. 13). Further, exogenous follistatin showed little effect as did the silencing construct on the number of cells within the inner cell mass (ICM), while recovering the relative number of lost trophectoderm (TE) cells and recovering total cell numbers (TOTAL) in culture (FIG. 14).

Thus, the exogenously added follistatin can enhance embryo quality demonstrated the recovery of TE cell numbers.

### Example 5

### This Example Demonstrates an Exemplary Follistatin Specific Negative Effect of Follistatin siRNA on TE Cell Numbers (a Marker of Embryo Quality) and Further Ablation Replacement Effects on Blastocyst mRNA Abundance for CDX2 (a Trophectoderm Cell Marker) and Lack of Effect on Inner Cell Mass (ICM) Cell Marker (Nanog)

Experiments such as those described in Example-4 (FIGS. 13 and 14) were initiated for determining relative RNA abundance of molecules associated with transplantation potential.

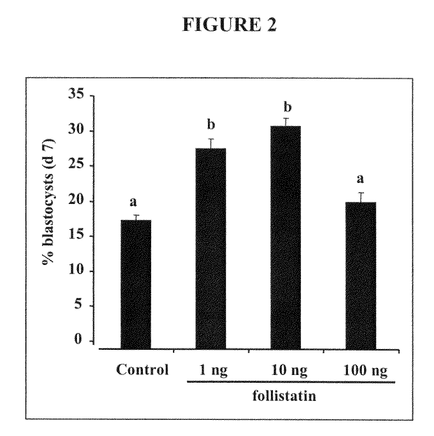

Recent research applied to the mouse has established that TE and ICM differentially express several lineage-specific transcription factors. Cdx2 becomes restricted to the TE and is required for TE formation (Yamanaka et al. 2006, Cell 126:663-676; FIG. 2). In contrast, Oct4 and Nanog become restricted to and influence ICM fate (Yamanaka et al. 2006, Cell 126:663-676; FIG. 2). The current understanding of their roles led to a model that predicts mutual antagonism between Oct4 and Cdx2 in supporting the formation of TE and ICM fates in the blastocyst (Yamanaka et al. 2006, Cell 126:663-676). Further, cdx2 is associated with successful (relatively higher) transplantation potential (Chawengsaksophak, et al., Proc Natl Acad Sci USA 2004; 101:7641-7645; Strumpf, et al. Development 2005; 132:2093-2102; Meissner, et al. Nature 2006; 439:212-215; each of which is herein incorporated by reference).

Nanog is a homeodomain transcription factor that is expressed specifically in undifferentiated embryonic stem (ES) cells and was shown to be essential in the maintenance of pluripotency in mouse ES cells. Knockdown experiments using NANOG small interfering (si) RNA resulted in induction of differentiation markers such as AFP, GATA4 and GATA6 for the endoderm and CDX2 for the trophectoderm. These results suggest that NANOG plays a crucial role in maintaining the pluripotent state of primate ES cells. NANOG over-expressing cell lines retained their undifferentiated state in the absence of a feeder layer, as shown by expression of undifferentiated ES cell markers such as alkaline phosphatase (ALP) and OCT-4 (Yasuda, et al., Genes Cells. 2006 September; 11(9):1115-23; herein incorporated by reference).

Further, biopsies of embryos that resulted in calf delivery were enriched with genes necessary for implantation (COX2 and CDX2), carbohydrate metabolism (ALOX15), growth factor (BMP15), signal transduction (PLAU), and placenta-specific 8 (PLAC8). Biopsies from embryos resulting in resorption are enriched with transcripts involved protein phosphorylation (KRT8), plasma membrane (OCLN), and glucose metabolism (PGK1 and AKR1B1). Biopsies from embryos resulting in no pregnancy are enriched with transcripts involved inflammatory cytokines (TNF), protein amino acid binding (EEF1A1), transcription factors (MSX1, PTTG1), glucose metabolism (PGK1, AKR1B1), and CD9, which is an inhibitor of implantation.

The caudal-related homeobox protein CDX2 is a transcriptional regulator essential for trophoblast lineage, functioning as early as implantation. The CDX2 gene is the earliest trophoblast-specific transcription factor reported to date (Roberts, et al., Reprod Biol Endocrinol 2: 47, 2004, Tolkunova, et al., Stem Cells 24: 139-144, 2006; each of which are herein incorporated by reference). An earlier gene targeting approach demonstrated that CDX2 null embryos fail to implant, suggestive of a major defect in TE development (Chawengsaksophak, et al., Nature 386:84-87, 1997, Rossant, Stem Cells, 19:477-482, 2001; each of which are herein incorporated by reference). The same authors showed that the implantation failure was due to loss of TE epithelial integrity and/or increased incidence of apoptosis of TE cells.

Therefore, CDX2 is one of the genes crucial for placental development, by which its aberrations in embryo can result in implantation or placental defect as reported by Hall et al. (Hall, et al., Reprod Fertil Dev 17: 261-261, 2005). See, Ashraf El-Sayed, et al., Physiol Genomics 28:84-96, 2006. First published Oct. 3, 2006; herein incorporated by reference.

Quantification of Oct-4 and CDX-2 mRNA in Bovine Blastocysts:

RNA isolation and quantitative real-time RT-PCR analysis of mRNA abundance for Nanog and CDX-2 was conducted according to procedures described in Bettegowda, et al., 2006, Mol Reprod Dev 73:267-278; Bettegowda, et al., 2007, Proceedings of the National Academy of Sciences USA 104:17602-17607; and Bettegowda, et al., 2008, Biology of Reproduction DOI:10.1095/biolreprod.107.067223; each of which are herein incorporated by reference. Briefly, total RNA was extracted from each of the blastocyst samples (n=4 pools of 2 blastocysts per pool for each treatment) using the RNAqueous micro kit (Ambion) according to manufacturer's instructions. The RNA was eluted twice using a 10 μl volume of prewarmed (75° C.) elution solution according to manufacturer's instructions. Residual genomic DNA in all extracted samples was removed by DNAse I digestion (Ambion). Total RNA (10 μl) from each sample for real-time RT-PCR analysis was utilized for reverse transcription (RT) using oligo dT₍₁₅₎ primers as described elsewhere. Embryos were evaluated for cleavage (Bettegowda et al., 2006, Mol Reprod Dev 73:267-278; herein incorporated by reference). After termination of cDNA synthesis, each RT reaction was then diluted with nuclease free water (Ambion) to a final volume of 100 μl. The quantification of all gene transcripts (Nanog, CDX2 and 18S rRNA) was done by real-time quantitative RT-PCR using SYBR Green PCR Master Mix (Applied Biosystems, Foster City, Calif.). Primers were designed for use using the Primer Express program (Applied Biosystems) and derived from bovine sequences found in GenBank (see Table 1). A primer matrix was performed for each gene tested to determine the optimal primer concentrations. Each reaction mixture consisted of 2 μl cDNA, 1.5 μl each of forward (5 μM) and reverse primers (5 μM), 7.5 μl nuclease-free water, and 12.5 μl SYBR Green PCR Master Mix in a total reaction volume of 25 μl (96-well plates). Reactions were performed in duplicate for each sample in an ABI prism 7000 Sequence Detection System (Applied Biosystems). For real-time RT-PCR experiments, the amounts of mRNAs of interest (CDX-2, Nanog) were normalized relative to abundance of an endogenous control (18S rRNA) to account for differences in total RNA concentrations between samples. The mean sample threshold cycle (CT) and mean endogenous control CT for each sample were calculated from duplicate wells. The relative amounts of target gene expression for each sample were calculated by using the formula 2—(ΔΔCT) as described elsewhere (Livak, et al., Methods 2001; 25:402-408; herein incorporated by reference). Effects of treatments on blastocyst mRNA abundance for CDX2 and Nanog was determined by one-way analysis of variance followed by Fisher's protected least significant difference test.

TABLE 1

| | Genebank | |
|---|---|---|
| | Accession | |
| Gene | number | PCR Primer Sequence |
| CDX-2 | AM293662 | F: 5′-FCGTCTGGAGCTGGAGAAGGA-3′ |
| | | R: 5′-CGGCCAGTTCGGCTTTC-3′ |
| Nanog | NM 001025344 | F: 5′-AAAGTTACGTGTCCTTGCAAACG-3′ |
| | | R: 5′-GAGGAGGGAAGAGGAGAGACAGT-3′ |

### Example 6

### Effect of Follistatin Treatment on Rhesus Monkey Embryonic Development

The inventors discovered that follistatin supplementation enhances the development of nuclear transfer embryos of primates.

### Methods:

In vitro fertilization (IVF) of rhesus monkey oocytes and culture of embryos was conducted according to previously described procedures (Vandevoort et al., J In Vitro Fert Embryo Transf 1989; 6: 85-91; VandeVoort et al., Theriogenology 2003; 59: 699-707; each of which are herein incorporated by reference).

After IVF, presumptive zygotes were cultured in Hamster embryo culture medium 9 plus PVA (HECM-9 PVA) for 48 h post insemination in the presence or absence of 10 ng/ml follistatin (n=6 replicates and 86 and 72 embryos total for control and follistatin treatments. Embryos were subsequently cultured in HECM-9 PVA containing 5% bovine calf serum (in the absence of exogenous follistatin).

Embryos were evaluated for cleavage at 30 hr. post insemination including counting the number of cleaved embryos and evaluated for blastocyte development at 48 and 188 hours post insemination (hpi).

The percent of embryos in culture which had cleaved by 30 h post insemination (hpi) were determined and further percent embryos reaching the blastocyst stage at 48 hpi and blastocyst development at approximately 188 hpi were determined.

The effect of follistatin treatment on % cleavage at 30 hpi and % blastocyst development was determined statistically by t test analysis.

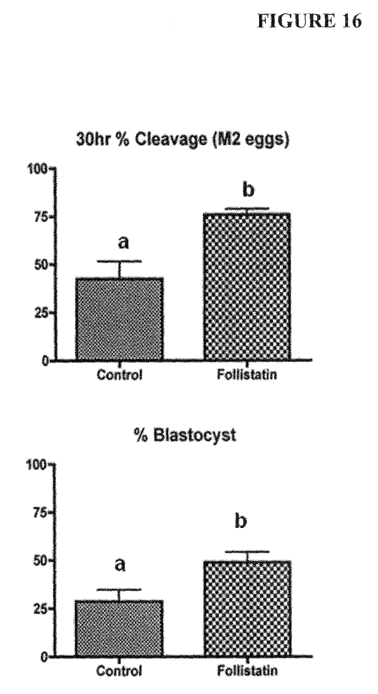

As shown in FIG. 16, follistatin supplementation significantly enhanced the development of rhesus monkey embryos. The progression of development of fertilized embryos to the M2 stage following cleavage was significantly increased (FIG. 16A). Further, the progression of development to the blastocyst stage was also significantly increased (FIG. 16B).

Therefore the inventors contemplate the use of follistatin supplementation in culture media for enhancing assisted reproductive techniques and stem cell development and further for using measurements of follistatin expression in diagnostic tests, as described herein, specifically for primates including humans.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A culture medium for in vitro culture of embryos comprising follistatin, wherein the development and survival of said embryos is enhanced when grown in said culture media compared to growth in culture media without follistatin.

2. The culture medium of claim 1, wherein said follistatin is present at a concentration from about 1 ng/ml to about 20 ng/ml.

3. The culture medium of claim 1, wherein said follistatin is present at a concentration of about 10 ng/ml.

4. The culture medium of claim 1, further comprising an embryo, wherein said embryo is selected from the group consisting of in vitro fertilization embryos, nuclear transfer embryos, cloned embryos, noncloned embryos, embryos for assisted reproductive techniques.

5. The culture medium of claim 1, wherein said embryos are mammalian embryos.

6. A kit comprising the culture medium of claim 5.

7. A method of increasing the survival of embryos, comprising,
a) providing,
i) an embryo,
ii) a culture medium, wherein said culture medium comprises follistatin,
b) culturing said embryo in a culture medium wherein the survival of said embryo is increased compared to survival of an embryo not grown in said media.

8. The method of claim 7, wherein said embryo is an in vitro fertilization embryo.

9. The method of claim 7, wherein said nuclear transfer embryo is a nuclear transfer embryo.

10. The method of claim 7, wherein said nuclear transfer embryo comprises genetic material obtained from a somatic cell.

11. The method of claim 7, wherein said follistatin is present at a concentration from about 1 ng/ml to about 20 ng/ml.

12. The method of claim 7, wherein said follistatin is present at a concentration of about 10 ng/ml.

13. The method of claim 7, wherein increased survival of said embryo is increasing the number of trophectoderm cells.

14. The method of claim 7, wherein said follistatin is a human recombinant follistatin.

15. The method of claim 7, wherein said method further comprises obtaining a biopsy from said embryo.

16. The method of claim 15, wherein said method further comprises c) determining the amount of follistatin expression in said embryo.

17. A method of generating stem cells comprising:
a) providing,
i) an embryo,
ii) a culture medium, wherein said culture medium comprises follistatin,
b) culturing said embryo in a culture medium to provide cultured embryonic cells,
c) generating stem cells from said cultured embryonic cells.

18. The method of claim 17, wherein culture of said embryo in said media generates an increased number of stem cells from said embryo as compared to the number of stem cells generated from an embryo not grown in said culture medium.

19. The method of claim 17, further comprising step d) harvesting said stem cells.

20. The method of claim 17, wherein said follistatin is present at a concentration from about 1 ng/ml to about 20 ng/ml.
